SEQUENCE LISTING (30 sequences)

SEQ ID NO: 1 - protein, 241 aa, Escherichia coli (strain K12)
MATNAKPVYKRILLKLSGEALQGTEGFGIDASILDRMAQEIKELVELGIQVGVVIGGGNLFRGAGLAKAGMNRVVGDHMGMLATVMNGLAMRDALHRAYVNARLMSAIPLNGVCDSYSWAEAISLLRNNRVVILSAGTGNPFFTTDSAACLRGIEIEADVVLKATKVDGVFTADPAKDPTATMYEQLTYSEVLEKELKVMDLAAFTLARDHKLPIRVFNMNKPGALRRVVMGEKEGTLITE

SEQ ID NO: 2 - protein, 808 aa, artificial
  engineered variant of Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 3 - protein, 808 aa, Bambusa oldhamii
MAAKLTRLHSLRERLSATFSSHPNELIALFSRYVHQGKGMLQRHQLLAEFDALIAADKEKYAPFEDILRAAQEAIVLPPWVALAIRPRPGVWDYIRVNVSELAVEELSVSEYLEFKEQLVDGHTNSNFVLELDFEPFNASFPRPSMSKSIGNGVQFLNRHLSSKLFQDKESLYPLLNFLKAHNHKGTTMMLNDRIQSLRGLQSALRKAEEYLMSFPQDTPYSEFNHRFQELGLEKGWGDTAKRVLDTIHLLLDLLEAPDPANLEKFLGTIPMTFNVVILSPHGYFAQSNVLGYPDTGGQVVYILDQVRALENEMLLRIKQQGLDITPKILIVTRLLPDAVGTTCGQRLEKVIGTEHTDILRVPFRTENGILRKWISRFDVWPFLETYTEDVANEIMREMQAKPDLIIGNYSDGNLVATLLAHKLGVTQCTIAHALEKTKYPNSDIYLDKFDSQYHFSCQFTADLIAMNHTDFIITSTFQEIAGSKDTVGQYESHIAFTLPGLYRVVHGIDVFDPKFNIVSPGADMSVYFPYTETDKRLTAFHPEIEELIYSDVENSEHKFVLKDKNKPIIFSMARLDRVKNMTGLVEMYGKNAHLRDLANLVIVAGDHGKESKDREEQAEFKRMYSLIDEYKLKGHIRWISAQMNRVRNGELYRYICDTKGVFVQPAFYEAFGLTVIESMTCGLPTIATCHGGPAEIIVDGVSGLHIDPYHSNKAADILVNFFEKCKEDPTYWDKISQGGLKRIYEKYTWKLYSERLMTLTGVYGFWKYVSNLERRETRRYLEMFYALKYRSLASAVPLAVDGDSAAN

SEQ ID NO: 4 - protein, 442 aa, artificial
  engineered viriant of Solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 5 - protein, 458 aa, Stevia rebaudiana
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 6 - protein, 275 aa, Neisseria meningitidis serogroup B (strain MC58)
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 7 - protein, 458 aa, artificial
  engineered variant of Stevia rebaudiana
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL

SEQ ID NO: 8 - protein, 351 aa, Arabidopsis thaliana
MMARNVLVSGGAGYIGSHTVLQLLLGGYSVVVVDNLDNSSAVSLQRVKKLAAEHGERLSFHQVDLRDRSALEKIFSETKFDAVIHFAGLKAVGESVEKPLLYYNNNLVGTITLLEVMAQHGCKNLVFSSSATVYGSPKEVPCTEEFPISALNPYGRTKLFIEEICRDVYGSDPEWKIILLRYFNPVGAHPSGDIGEDPRGIPNNLMPFVQQVAVGRRPHLTVFGNDYNTKDGTGVRDYIHVIDLADGHIAALRKLEDCKIGCEVYNLGTGNGTSVLEMVDAFEKASGKKIPLVIAGRRPGDAEVVYASTERAESELNWKAKYGIEEMCRDLWNWASNNPYGYDSSSEDNSH

SEQ ID NO: 9 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLKFLRLHSHQGKNLMLSEKIQNLNTLQHTLRKAEEYLAELKSETLYEEFEAKFEEIGLERGWGDNAERVLDMIRLLLDLLEAPDPCTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENKEHLCVLKDKKKPILFTMARLDRVKNLSGLVEWYGKNTRLRELANLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADTLADFFTKCKEDPSHWDEISKGGLQRIEEKYTWQIYSQRLLTLTGVYGFWKHVSNLDRLEARRYLEMFYALKYRPLAQAVPLAQDD

SEQ ID NO: 10 - protein, 241 aa, Salmonella enterica
MATNAKPVYKRILLKLSGEALQGTEGFGIDASILDRMAQEIKELVELGIQVGVVIGGGNLFRGAGLAKAGMNRVVGDHMGMLATVMNGLAMRDALHRAYVNARLMSAIPLNGVCDNYSWAEAISLLRNNRVVILSAGTGNPFFTTDSAACLRGIEIEADVVLKATKVDGVFTADPAKDPSATMYDQLTYSEVLDKELKVMDLAAFTLARDHKLPIRVFNMNKPGALRRVVMGEKEGTLITE

SEQ ID NO: 11 - protein, 240 aa, Bacillus subtilis
MEKPKYKRIVLKLSGEALAGEQGNGINPTVIQSIAKQVKEIAELEVEVAVVVGGGNLWRGKTGSDLGMDRATADYMGMLATVMNSLALQDSLETLGIQSRVQTSIEMRQVAEPYIRRKAIRHLEKKRVVIFAAGTGNPYFSTDTTAALRAAEIEADVILMAKNNVDGVYNADPRKDESAVKYESLSYLDVLKDGLEVMDSTASSLCMDNDIPLIVFSIMEEGNIKRAVIGESIGTIVRGK

SEQ ID NO: 12 - protein, 235 aa, Ureaplasma parvum
MRKQRIVIKISGACLKQNDSSIIDFIKINDLAEQIEKISKKYIVSIVLGGGNIWRGSIAKELDMDRNLADNMGMMATIINGLALENALNHLNVNTIVLSAIKCDKLVHESSANNIKKAIEKEQVMIFVAGTGFPYFTTDSCAAIRAAETESSIILMGKNGVDGVYDSDPKINPNAQFYEHITFNMALTQNLKVMDATALALCQENNINLLVFNIDKPNAIVDVLEKKNKYTIVSK

SEQ ID NO: 13 - protein, 225 aa, Pyrococcus furiosus
MRIVFDIGGSVLVPENPDIDFIKEIAYQLTKVSEDHEVAVVVGGGKLARKYIEVAEKFNSSETFKDFIGIQITRANAMLLIAALREKAYPVVVEDFWEAWKAVQLKKIPVMGGTHPGHTTDAVAALLAEFLKADLLVVITNVDGVYTADPKKDPTAKKIKKMKPEELLEIVGKGIEKAGSSSVIDPLAAKIIARSGIKTIVIGKEDAKDLFRVIKGDHNGTTIEP

SEQ ID NO: 14 - protein, 239 aa, Neisseria meningitidis
MTQQIKYKRVLLKLSGESLMGSDPFGINHDTIVQTVGEIAEVVKMGVQVGIVVGGGNIFRGVSAQAGSMDRATADYMGMMATVMNALALKDAFETLGIKARVQSALSMQQIAETYARPKAIQYLEEGKVVIFAAGTGNPFFTTDTAAALRGAEMNCDVMLKATNVDGVYTADPKKDPSATRYETITFDEALLKNLKVMDATAFALCRERKLNIVVFGIAKEGSLKRVITGEDEGTLVHC

SEQ ID NO: 15 - protein, 242 aa, Streptococcus pyogenes
MEPKYQRILIKLSGEALAGEKGVGIDIPTVQAIAKEIAEVHVSGVQIALVIGGGNLWRGEPAADAGMDRVQADYTGMLGTVMNALVMADSLQHYGVDTRVQTAIPMQNVAEPYIRGRALRHLEKNRIVVFGAGIGSPYFSTDTTAALRAAEIEADAILMAKNGVDGVYNADPKKDANAVKFDELTHGEVIKRGLKIMDATASTLSMDNDIDLVVFNMNEAGNIQRVVFGEHIGTTVSNKVCD

SEQ ID NO: 16 - protein, 237 aa, Haemophilus influenzae
MSQPIYKRILLKLSGEALQGEDGLGIDPAILDRMAVEIKELVEMGVEVSVVLGGGNLFRGAKLAKAGMNRVVGDHMGMLATVMNGLAMRDSLFRADVNAKLMSAFQLNGICDTYNWSEAIKMLREKRVVIFSAGTGNPFFTTDSTACLRGIEIEADVVLKATKVDGVYDCDPAKNPDAKLYKNLSYAEVIDKELKVMDLSAFTLARDHGMPIRVFNMGKPGALRQVVTGTEEGTTIC

SEQ ID NO: 17 - protein, 240 aa, Xanthomonas campestris
MSELSYRRILLKLSGEALMGDGDYGIDPKVINRLAHEVIEAQQAGAQVALVIGGGNIFRGAGLAASGMDRVTGDHMGMLATVINALAMQDALEKLGAKVRVMSAIKINDVCEDFIRRRAIRHLEKGRIAIFAAGTGNPFFTTDSGAALRAIEIGADLLLKATKVDGVYDKDPKKHSDAVRYDSLTYDEVIMQGLEVMDTAAFALARDSDLPLRIFGMSEPGVLLRILHGAQIGTLVQGRS

SEQ ID NO: 18 - protein, 226 aa, Saccharolobus solfataricus
MNIILKISGKFFDEDNVDNLIVLRQSIKELADNGFRVGIVTGGGSTARRYIKLAREIGIGEAYLDLLGIWASRLNAYLVMFSLQDLAYMHVPQSLEEFIQDWSHGKVVVTGGFQPGQSTAAVAALVAEASSSKTLVVATNVDGVYEKDPRIYADVKLIPHLTTQDLRKILEGSQSVQAGTYELLDPLAIKIVERSKIRVIVMNYRKLNRIIDILKGEEVSSIIEPV

SEQ ID NO: 19 - protein, 261 aa, Mycobacterium tuberculosis
MTEPDVAGAPASKPEPASTGAASAAQLSGYSRVLLKLGGEMFGGGQVGLDPDVVAQVARQIADVVRGGVQIAVVIGGGNFFRGAQLQQLGMERTRSDYMGMLGTVMNSLALQDFLEKEGIVTRVQTAITMGQVAEPYLPLRAVRHLEKGRVVIFGAGMGLPYFSTDTTAAQRALEIGADVVLMAKAVDGVFAEDPRVNPEAELLTAVSHREVLDRGLRVADATAFSLCMDNGMPILVFNLLTDGNIARAVRGEKIGTLVTT

SEQ ID NO: 20 - protein, 240 aa, Helicobacter pylori
MQAKIKNKRVLVKFSGEALAGDNQFGIDIHVLDHIAKEIKSLVENDIEVGIVIGGGNIIRGVSAAQGGIIRRTSGDYMGMLATVINAVAMQEALEHIGLDTRVQSAIEIKEICESYIYRKAIRHLEKGRVVIFGAGTGNPFFTTDTAATLRAIEIGSDLIIKATKVDGIYDKDPNKFKDAKKLDTLSYNDALIGDIEVMDDTAISLAKDNKLPIVVCNMFKKGNLLQVIKHQQGVFSMVK

SEQ ID NO: 21 - protein, 202 aa, Arabidopsis thaliana [sequence omitted]

SEQ ID NO: 22 - protein, 196 aa, Homo sapiens [sequence omitted]

SEQ ID NO: 23 - protein, 204 aa, Saccharomyces cerevisiae
MTAATTSQPAFSPDQVSVIFVLGGPGAGKGTQCEKLVKDYSFVHLSAGDLLRAEQGRAGSQYGELIKNCIKEGQIVPQEITLALLRNAISDNVKANKHKFLIDGFPRKMDQAISFERDIVESKFILFFDCPEDIMLERLLERGKTSGRSDDNIESIKKRFNTFKETSMPVIEYFETKSKVVRVRCDRSVEDVYKDVQDAIRDSL

SEQ ID NO: 24 - protein, 228 aa, Canis lupus
MVSRCGRRLLHVLGLSFPLLTRPPLFVCPRRLMKPLVVFVLGGPGAGKGTQCARIVEKYGYTHLSAGELLRDERKNPASQYGELIEKYIKDGKIVPVEITISLLKREMDQTMAANAQKNKFLIDGFPRNQDNLQGWNKTMDGKADVSFVLFFDCNNEICIERCLERGKSSGRSDDNRESLEKRIQTYLQSTKPIIDLYEEMGKVKKIDASKSVDEVFDEVVKIFDKEG

SEQ ID NO: 25 - protein, 228 aa, Gorilla gorilla gorilla
MLSRCRSRLLHVLGLSFLLQTRRPILLCSPRLMKPLVVFVLGGPGAGKGTQCARIVEKYGYTHLSAGELLRDERKNPDSQYGELIEKYIKEGKIVPVEITISLLKREMDETMAANAQKNKFLIDGFPRNQDNLQGWNKTMDGKADVSFVLFFDCNNEICIERCLERGKSSGRSDDNRESLEKRIQTYLQSTKPIIDLYEEMGKVKKIDASKSVDEVFDEVVQIFDKEG

SEQ ID NO: 26 - protein, 227 aa, Rattus norvegicus
MLSSCRRWLLHVLVPSFPPLTRGLRFFPLQLMKPLVVFVLGGPGAGKGTQCARIVEKYGYTHLSAGELLRDERKNPDSQYGELIEKYIKEGKIVPVEITISLLKREMDQTMAANAQKNKFLIDGFPRNQDNLQGWNKTMDGKADVSFVLFFDCNNEICIDRCLERGKSSGRSDDNRESLEKRIQTYLESTKPIIDLYEEMGKVKKIDASKSVDEVFGDVMKIFDKEG

SEQ ID NO: 27 - protein, 227 aa, Vulpes vulpes
MLSSCRRLLPHVLVPSCPPLTRRLWLFPLHLMKPLVVFVLGGPGAGKGTQCARIVEKYGYTHLSAGELLRDERKNPDSQYGELIEKYIKEGKIVPVEITISLLKREMDQTMAANAQKNKFLIDGFPRNQDNLQGWNKTMDGKADVSFVLFFDCNNEICIERCLERGKSSGRSDDNRESLEKRIQTYLESTKPIIDLYEEMGKVKKIDASKSVDEVFGEVVKIFDKEG

SEQ ID NO: 28 - protein, 228 aa, Dictyostelium discoideum
MLSRCGRRLLHVLGLSFPLLTRPPLFLCPRRLMKPLVVFVLGGPGAGKGTQCARIVEKYGYTHLSAGELLRDERKNPASQYGELIEKYIKDGKIVPVEITISLLKREMDQTMAANAQKNKFLIDGFPRNQDNLQGWNKTMDGKADVSFVLFFDCNNEICIERCLERGKSSGRSDDNRESLEKRIQTYLQSTKPIIDLYEEMGKVKKIDASKSVDEVFDEVVKIFDKEG

SEQ ID NO: 29 - protein, 339 aa, Morchella conica
MSAIRSSVSGLARTFKLNSLPALRVSRLHTIPSAAAPHSPTLPTSRISPGSFRQGLTHSKFGSSQGQCRLYAAGPRNLGETGGSKNPFPVVPLVLIFTAGSGGFYLLVNSRKETMPATEKTLPRSPKKAKTTPTFSPKDVSVIFVLGGPGAGKGTQCQNLVRDYGFVHLSAGDLLRAEQNRDGSEFGELIKTYIREGRIVPMEVTVALLENAMKESIANGQNRFLIDGFPRQMDQAIKFETEVCPSVFTLFFDCPEEVMLERLLKRGETSGREDDNIESIKKRFRTFVNTSMPVVDMFEKEGKVVRVDCTQGVEEVYSAVKKEVSSKFKPMKEKAVMTA

SEQ ID NO: 30 - protein, 318 aa, Tuber magnatum
MSSAFRSISGRIVSNLQHPIRNCPAALTRLHLRFQPRNVQPSSIPSAFRGAPARNYATGSGKGGETAGSKNPFPIIPLLAILAAGSGGFYFLVQSRKEPMPAIEEPSRKPRLTAGDVKKPAFGPGEVSVIFILGGPGAGKGTQCQNLVRDYGFVHLSAGDLLRAEQNREGSEFGELIKTYIREGKIVPMEVTVALLENAMRDAIAAQQKTMFLIDGFPRQMDQALKFEEEVSESRFTLFFECSEDTMLQRLLKRGETSGRIDDNIESIKKRFHTFQVTSMPVVDYYDKKGKVVRIDASSTREGVYSQVQKQLIDKLKA

(19) United States
(12) Patent Application Publication
(10) Pub. No.: US 2023/0304055 A1
(43) Pub. Date: Sep. 28, 2023

(54) ONE-POT CELL-FREE GLYCOSYLATION PROCESS

(71) Applicant: C-LEcta GmbH, Leipzig (DE)

(72) Inventors: Andreas VOGEL, Leipzig (DE); Claudia FELLER, Leipzig (DE); Christopher David BAYER, Amsterdam (NL); Gregor HOFFMANN, Markkleeberg (DE); Stefan SCHOENERT, Leipzig (DE)

(73) Assignee: C-LEcta GmbH, Leipzig (DE)

(21) Appl. No.: 18/011,258

(22) PCT Filed: Jun. 29, 2021
(86) PCT No.: PCT/EP2021/067816

§ 371 Date: Dec. 19, 2022

(30) Foreign Application Priority Data
Jul. 3, 2020 (EP) 20183991.7
Jul. 3, 2020 (EP) 20183993.3

(51) Int. Cl. C12P 19/18; C12P 19/56; C12P 19/44

(52) U.S. Cl. CPC C12P 19/18; C12P 19/56; C12P 19/44

(57) ABSTRACT

The invention relates to a process for the preparation of a glycosylated organic compound by in vitro glycosylation of an organic compound having a nucleophilic group with a saccharide under catalysis of a Leloir glycosyltrans-ferase system comprising at least a first glycosyl transferase and a second glycosyl transferase. The invention further relates to a composition comprising a glycosylated organic compound that is obtainable by the process according to the invention.

Priority is claimed of European patent application no. 20183991.7 and European patent application no. 20183993.3, both filed on Jul. 3, 2020.

The invention relates to a process for the preparation of a glycosylated organic compound by in vitro glycosylation of an organic compound having a nucleophilic group with a saccharide under catalysis of a Leloir glycosyltransferase system comprising at least a first glycosyl transferase and a second glycosyl transferase. The invention further relates to a composition comprising a glycosylated organic compound that is obtainable by the process according to the invention.

The possibility to selectively append glycosyl functionalities on organic molecules paves the way towards efficient synthesis of a variety of functional building blocks and key intermediates which are of high relevance for diverse industrial applications.

Particularly, implementation of Leloir-type glycosyltransferases in such processes allows for efficient glycosylation in a protecting group free setting and thus has been drawing increasing attention. Such enzyme catalyze the transfer of a glycosyl moiety from an activated sugar nucleotide diphosphate in an exergonic reaction delivering the glycosylated organic acceptor and a nucleotide diphosphate (NDP). In this regard, a major bottleneck is represented by the limited availability of the nucleotide-activated sugar substrates which needs to be continuously regenerated for otherwise the costs related to the employment of the starting material in stoichiometric amount would prevent the practical feasibility of the process.

A number of approaches where therefore developed to provide for efficient in-situ regeneration of the sugar nucleotide diphosphates (NDP-sugars). One of such approaches relies on the transfer of a nucleotide unit on a phosphor-sugar under catalysis of a nucleotidyltransferases which might however present limitations in that it requires employment of three or more enzymes. Alternatively, the necessary NDP-sugar might be generated exploiting a synthase route using a sucrose synthase (SuSy) by virtue of which, the NDP-sugar starting material can be readily accessed starting from NDP and sucrose.

The application of Leloir glycosyltransferase/NDP based systems for the synthesis of glycosylated organic substrates is known in the art.

WO 2009 015268 A2 relates to glycosyltransferases possessing expanded substrate specificities and their use in enzymatic synthesis of glycosylated compounds with novel and/or improved bioactivities.

WO 2013 176738 A1 relates to methods of preparing highly purified steviol glycosides by means of a catalyst system which comprising a UDP-glucosyltransferase. The application teaches that employment of a sucrose synthase allows for recycling the UDP-glucose and thus to use UDP in catalytic amount.

WO 2017 093895 A1, WO 2018 21327 9A1 and WO 2020 028039 A1 relate to methods producing steviol glycosides and/or rebaudioside derivatives involving subjecting the starting material, such as rebaudioside A to a UDP-glucosyl transferase enzyme. The disclosure teaches that a sucrose synthase can be implemented in the system thus allowing for UDP regeneration.

WO 2018 144675 A1 provides engineered glycosyltransferase (GT) enzymes, polypeptides having GT activity, and method for performing glycosylation reactions relying on the transfer of glycosyl residues from a nucleotide diphosphate activated sugar donor to an organic acceptor.

DE 19 516 952 A1 describes an enzymatic method for synthesis of the keto sugar dUDP-6-desoxy-D-xylo-4-hexulose from dUMP, and the subsequent use of such keto sugar (or comparable keto sugars or keto sugar derivatives) in a separate enzymatic galactosylation of mono- and oligosaccharides catalyzed by sucrose synthase, β-1-4 galactosyltransferase and uridine diphosphate glucose 4′-epimerase (UDP-glucose 4′-epimerase) under in situ regeneration of the nucleotide sugar (i. e. the Nucleoside diphosphate sugar), wherein the keto sugar (or keto sugar derivative) acts as activator and reduces epimerase enzyme inhibition through UMP, UDP, UDP Galactose or UDP Glucose.

CN 110 699 373 A relates to high-yielding uridine diphosphate glucose strain transformed in Escherichia coli to overexpress relevant key enzymes in the pyrimidine synthesis pathway such as orotate pyrophosphorylase pyrE, orotate nucleotide decarboxylase pyrF and uracil nucleotide kinase pyrH.

Due to the use of expansive starting material such as costly NDPs, the processes of the prior art are not satisfactory in every respect and are not suitable for the production of glycosylated organic compounds at industrial scale.

It is an object of the invention to provide improved processes for the enzymatic synthesis of glycosylated organic compounds. It is further an object of the invention to provide a cost-efficient enzymatic glycosylation process relying on the use of cheaper starting materials. It is further an object of the invention to provide an enzymatic glycosylation process in which the phosphate donor is not regenerated widely applicable on industrial scale.

One or more of these objects have been achieved by the subject-matter of the patent claims

It has been surprisingly found that the process according to the invention affords high product yield while relying on the use of nucleoside monophosphate starting materials which, as compared to their NDPs counterparts, are far cheaper. The invention provides therefore a more scalable and cost-efficient glycosylation platform as compared to the process disclosed in the prior art. The invention can be realized concurrently with glycosylation process as described in the prior art without any process stops or separate processing steps or vessels and without interfering with process yields.

A first aspect of the invention relates to a process for the preparation of a glycosylated organic compound by in vitro glycosylation of an organic compound having a nucleophilic group with a saccharide under catalysis of a Leloir glycosyltransferase system comprising at least a first glycosyl transferase and a second glycosyl transferase, the process comprising the steps of:
(a) providing a nucleoside monophosphate and a phosphate donor;
(b) reacting the nucleoside monophosphate and the phosphate donor provided in step (a) under catalysis of a nucleoside monophosphate kinase thereby obtaining a nucleoside diphosphate;
(c) providing a saccharide donor;
(d) reacting the nucleoside diphosphate obtained in step (b) with the saccharide of the saccharide donor provided in step (c) under catalysis of the first glycosyl transferase thereby obtaining a nucleoside diphosphate saccharide;
(e) providing the organic compound having a nucleophilic group; and
(f) reacting the nucleoside diphosphate saccharide obtained in step (d) with the organic compound having a nucleophilic group provided in step (e) under catalysis of at least the second glycosyltransferase thereby obtaining the glycosylated organic compound.

The process according to the invention is directed to the preparation of a glycosylated compound by in vitro glycosylation of an organic compound having a nucleophilic group with a saccharide under catalysis of a Leloir glycosyltransferase system comprising at least a first glycosyl transferase and a second glycosyl transferase.

For the purpose of the specification, a Leloir glycosyltransferase system is preferably regarded as a catalyst system comprising at least a first glycosyltransferase and a second glycosyltransferase. For the purpose of the specification, all glycosyltransferases are preferably to be regarded as sugar nucleotide-dependent (Leloir) glycosyltransferases, i.e. enzymes which are able to catalyze the transfer of a glycosyl moiety of a phosphor-containing nucleotide sugar substrate to a nucleophilic group of an acceptor.

The catalyst system comprising at least a first glycosyltransferase and a second glycosyltransferase may consist of the first glycosyltransferase and the second glycosyltransferase, or alternatively comprise one or more additional glycosyltransferases. Further, the two glycosyltransferases are not necessarily separate physical entities. Rather, it is contemplated according to the present invention that a single glycosyltransferase (i.e. bi- or polyfunctional enzyme) capable of fulfilling the function of at least both the first glycosyltransferase and second glycosyltransferase is equally suited.

It has been surprisingly found that the catalyst system of at least a first glycosyltransferase and a second glycosyltransferase can be expanded by a kinase enzyme catalyzing the production of a nucleoside diphosphate starting from a nucleoside monophosphate and a phosphate donor without disturbances of the overall reaction or of the reaction efficiency in formation of the desired glycosylated organic compound or otherwise interfering with the process. In particular, it has been found surprising that the enzymes can act concomitantly, or simultaneously, and in one step without creating imbalance in the reaction efficiency and that the reaction does not need to be run sequentially, by applying interim process stops or purification steps, or separate vessels.

In step (a) of the process according to the invention, a nucleoside monophosphate and a phosphate donor are provided.

For the purpose of the specification, a nucleoside monophosphate is preferably a phosphoester or an organic compound; preferably a monophosphorylated conjugate, of a ribose or a deoxyribose that is linked to a nucleobase selected from the group consisting of adenine, guanine, inosine, cytosine, thymine, and uracil.

Preferably, the nucleoside monophosphate is not adenosine monophosphate.

Preferably, the nucleoside monophosphate is a monophosphorylated conjugate of a ribose or a deoxyribose with a pyrimidine base selected from the group consisting of cytosine, thymine, and uracil.

Preferably, the nucleoside monophosphate is a monophosphorylated conjugate of a ribose or a deoxyribose with uracil most preferably uridine monophosphate.

Preferably, the nucleoside monophosphate is employed in a concentration of at least 0.012 mM; preferably at least 0.05 mM; more preferably at least 0.1 mM.

Preferably, the nucleoside monophosphate is employed in a concentration of at most 4.0 mM; preferably at most 3.0 mM; more preferably at most 2.0 mM.

Preferably, the nucleoside monophosphate is employed in a concentration within the range of 0.382±0.37 mM, or 0.4±0.37 mM, or 0.46±0.37 mM, or 0.5±0.37 mM, or 0.56±0.37 mM, or 0.62±0.37 mM, or 0.68±0.37 mM, or 0.74±0.37 mM, or 0.8±0.37 mM, or 0.88±0.37 mM, or 1.48±0.37 mM, or 1.48±0.37 mM, or 1.63±0.37 mM, or 1.78±0.37 mM, or 1.93±0.37 mM, or 2.08±0.37 mM, or 2.23±0.37 mM, or 2.38±0.37 mM, or 2.53±0.37 mM, or 2.68±0.37 mM, or 2.83±0.37 mM, or 2.98±0.37 mM, or 3.13±0.37 mM, or 3.28±0.37 mM, or 3.43±0.37 mM, or 3.58±0.37 mM, or 3.63±0.37 mM; preferably within the range of 0.382±0.27 mM, or 0.4±0.27 mM, or 0.46±0.27 mM, or 0.5±0.27 mM, or 0.56±0.27 mM, or 0.62±0.27 mM, or 0.68±0.27 mM, or 0.74±0.27 mM, or 0.8±0.27 mM, or 0.88±0.27 mM, or 1.48±0.27 mM, or 1.48±0.27 mM, or 1.63±0.27 mM, or 1.78±0.27 mM, or 1.93±0.27 mM, or 2.08±0.27 mM, or 2.23±0.27 mM, or 2.38±0.27 mM, or 2.53±0.27 mM, or 2.68±0.27 mM, or 2.83±0.27 mM, or 2.98±0.27 mM, or 3.13±0.27 mM, or 3.28±0.27 mM, or 3.43±0.27 mM, or 3.58±0.27 mM, or 3.63±0.27 mM; more preferably within the range of 0.382±0.27 mM, or 0.4±0.17 mM, or 0.46±0.17 mM, or 0.5±0.17 mM, or 0.56±0.17 mM, or 0.62±0.17 mM, or 0.68±0.17 mM, or 0.74±0.17 mM, or 0.8±0.17 mM, or 0.88±0.17 mM, or 1.48±0.17 mM, or 1.48±0.17 mM, or 1.63±0.17 mM, or 1.78±0.17 mM, or 1.93±0.17 mM, or 2.08±0.17 mM, or 2.23±0.17 mM, or 2.38±0.17 mM, or 2.53±0.17 mM, or 2.68±0.17 mM, or 2.83±0.17 mM, or 2.98±0.17 mM, or 3.13±0.17 mM, or 3.28±0.17 mM, or 3.43±0.17 mM, or 3.58±0.17 mM, or 3.63±0.17 mM.

For the purpose of the specification a phosphate donor is preferably as a phosphoryl-containing compound; preferably selected from the group comprising organic monophosphates, organic polyphosphates, inorganic monophosphates and inorganic polyphosphates which is capable of donating a phosphate unit during the course of a reaction.

Preferably, the phosphate donor is selected from the group consisting of organic monophosphates, organic polyphosphates, inorganic monophosphates and inorganic polyphosphates.

Preferably, the phosphate donor is selected from the group consisting of nucleoside monophosphates, nucleoside polyphosphates, creatine monophosphate, creatine polyphosphate, and phosphoenolpyruvic acid.

Preferably, the phosphate donor is a mono- or polyphosphate of a nucleoside; preferably a nucleoside triphosphate; more preferably adenosine triphosphate.

Preferably, the phosphate donor is employed in a concentration which is at least as high as the concentration of the nucleoside monophosphate.

Preferably, the phosphate donor is employed in a concentration that is relatively at least 0.1 mM greater than the concentration of the nucleoside monophosphate.

Preferably, the phosphate donor is employed in a concentration of at least 0.012 mM; preferably at least 0.05 mM; more preferably at least 0.1 mM.

Preferably, the phosphate donor is employed in a concentration of at most 4.0 mM; preferably at most 3.0 mM; more preferably at most 2.0 mM.

Preferably, the phosphate donor is employed in a concentration within the range of 0.382±0.37 mM, or 0.4±0.37 mM, or 0.46±0.37 mM, or 0.5±0.37 mM, or 0.56±0.37 mM, or 0.62±0.37 mM, or 0.68±0.37 mM, or 0.74±0.37 mM, or 0.8±0.37 mM, or 0.88±0.37 mM, or 1.48±0.37 mM, or 1.48±0.37 mM, or 1.63±0.37 mM, or 1.78±0.37 mM, or 1.93±0.37 mM, or 2.08±0.37 mM, or 2.23±0.37 mM, or 2.38±0.37 mM, or 2.53±0.37 mM, or 2.68±0.37 mM, or 2.83±0.37 mM, or 2.98±0.37 mM, or 3.13±0.37 mM, or 3.28±0.37 mM, or 3.43±0.37 mM, or 3.58±0.37 mM, or 3.63±0.37 mM; preferably within the range of 0.382±0.27 mM, or 0.4±0.27 mM, or 0.46±0.27 mM, or 0.5±0.27 mM, or 0.56±0.27 mM, or 0.62±0.27 mM, or 0.68±0.27 mM, or 0.74±0.27 mM, or 0.8±0.27 mM, or 0.88±0.27 mM, or 1.48±0.27 mM, or 1.48±0.27 mM, or 1.63±0.27 mM, or 1.78±0.27 mM, or 1.93±0.27 mM, or 2.08±0.27 mM, or 2.23±0.27 mM, or 2.38±0.27 mM, or 2.53±0.27 mM, or 2.68±0.27 mM, or 2.83±0.27 mM, or 2.98±0.27 mM, or 3.13±0.27 mM, or 3.28±0.27 mM, or 3.43±0.27 mM, or 3.58±0.27 mM, or 3.63±0.27 mM; more preferably within the range of 0.382±0.27 mM, or 0.4±0.17 mM, or 0.46±0.17 mM, or 0.5±0.17 mM, or 0.56±0.17 mM, or 0.62±0.17 mM, or 0.68±0.17 mM, or 0.74±0.17 mM, or 0.8±0.17 mM, or 0.88±0.17 mM, or 1.48±0.17 mM, or 1.48±0.17 mM, or 1.63±0.17 mM, or 1.78±0.17 mM, or 1.93±0.17 mM, or 2.08±0.17 mM, or 2.23±0.17 mM, or 2.38±0.17 mM, or 2.53±0.17 mM, or 2.68±0.17 mM, or 2.83±0.17 mM, or 2.98±0.17 mM, or 3.13±0.17 mM, or 3.28±0.17 mM, or 3.43±0.17 mM, or 3.58±0.17 mM, or 3.63±0.17 mM.

In step (b) of the process according to the invention, the nucleoside monophosphate and the phosphate donor provided in step (a) are reacted under catalysis of a nucleoside monophosphate kinase thereby obtaining a nucleoside diphosphate.

For the purpose of the specification a nucleoside monophosphate kinase is preferably as an enzyme able to catalyze the transfer of a phosphate unit from a phosphate donor to the phosphoryl group nucleoside monophosphate.

Preferably, the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase)

In a preferred embodiment the nucleoside monophosphate kinase is an uridylate kinase (UMP-kinase) belonging to EC class 2.7.4.22.

In other preferred embodiments the nucleoside monophosphate kinase is an uridylate kinase (UMP-kinase) belonging to EC class EC 2.7.4.14.

UMP Kinases in the meaning of the invention shall mean kinase enzymes, that preferentially catalyze the phosphorylation of a uridine nucleoside monophosphate by use of a phosphate donor. UMP kinase in the meaning of the invention shall also comprise kinase enzymes that catalyze the phosphorylation of other nucleoside monophosphate than UMP by use of a phosphate donor with either lower or higher efficiency or specificity.

Preferably the nucleoside monophosphate kinase is an uridylate kinase (UMP-kinase) comprising or consisting of an amino acid sequence selected from the group of sequences consisting of SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

Preferably the nucleoside monophosphate kinase is an uridylate kinase (UMP-kinase) comprising or consisting of an amino acid sequence corresponding to SEQ ID NO: 1.

Preferably the nucleoside monophosphate kinase is an uridylate kinase (UMP-kinase) belonging to EC class EC 2.7.4.14 and preferably comprises or consists of an amino acid sequence selected from the group of sequences consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19.

Preferably the nucleoside monophosphate kinase is an uridylate kinase (UMP-kinase) belongs to E.C class 2.7.4.22 and preferably comprises or consists of an amino acid sequence selected from the group of sequences consisting of SEQ ID NO: 1, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30.

Preferably the nucleoside monophosphate kinase is an uridylate kinase (UMP-kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 10.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 11.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 12.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 13.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 14.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 15.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 16.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 17.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 18.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 19.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 20.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 21.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 22.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 23.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 24.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 25.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 26.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 27.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 28.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 29.

Preferably the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 30.

The nucleoside monophosphate kinase according to the invention comprises such an amino acid sequence with a defined identity to any of the amino acid sequences selected from the group consisting of SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30. This means that for any of the nucleoside monophosphate kinases according to the invention an individual nucleoside monophosphate kinase may comprise said amino acid sequences as a subsequence of its overall amino acid sequence, or that the nucleoside monophosphate kinase according to the invention may essentially consist of said amino acid sequence. When such individual nucleoside monophosphate kinase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the nucleoside monophosphate kinase is to be immobilized on a solid support, e.g. for purification purposes.

Preferably, the nucleoside monophosphate kinase according to the invention comprises such an amino acid sequence with a defined identity to any of the amino acid sequences selected from the groups consisting of sequences
Group 1: SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19 wherein the nucleoside monophosphate kinase preferably belongs to EC class EC 2.7.4.14; or
Group 2: SEQ ID NO: 1, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29 and SEQ ID NO: 30, wherein the nucleoside monophosphate kinase preferably belongs to EC class 2.7.4.22.

This means that the nucleoside monophosphate kinase according to the invention may comprise said amino acid sequence as a subsequence of its overall amino acid sequence, or that the nucleoside monophosphate kinase according to the invention may essentially consist of said amino acid sequence. When the nucleoside monophosphate kinase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the nucleoside monophosphate kinase is to be immobilized on a solid support, e.g. for purification purposes.

In the meaning of this invention, the Percent identity is calculated as: Sequence Identity [%]=number of Matches/L×100, wherein L is the number of aligned positions, i.e. identities and nonidentities (including gaps, if any). Identity is preferably calculated using BLASTP (see, for example, Altschul S F et al. (1997) “Gapped BLAST and PSI-BLAST: a new generation of protein database search programs”, Nucleic Acids Res. 25:3389-3402; or Altschul S F (2005) “Protein database searches using compositionally adjusted substitution matrices.” FEBS J. 272:5101-5109); preferably with the following algorithm parameters: Matrix: BLOSUM62; Gap Costs: Existence: 11 Extension: 1, Expect threshold: 10 and Word size: 6. Results are filtered for sequences with more than 35% query coverage. BlastP can be accessed online at the NCBI Homepage (https://blast.ncbi.nlm.nih.gov/Blast.cgi?PROGRAM=blastp&PAGE_TYPE=BlastSearch&LINK_LOC=blasthome). Other program settings can be adjusted as desired, for example using the following settings:
Field “Enter Query Sequence”: Query subrange: none;
Field “Choose Search Set”: Database: non-redundant protein sequences (nr); optional parameters: none
Field “Program Selection”: Algorithm: blastp (protein-protein BLAST);
Algorithm parameters: Field “General parameters”: Max target sequences: 20000; Short queries: Automatically adjust parameters for short input sequences; Expect threshold: 10; Word size: 6; Max matches in a query range: 0;
Algorithm parameters: Field “Scoring parameters”: Matrix: BLOSUM62; Gap Costs: Existence: 11 Extension: 1; Compositional adjustments: Conditional compositional score matrix adjustment;
Algorithm parameters: Field “Filters and Masking”: Filter: none; Mask: none.

In addition to the default parameters for calculation of Percent identity in the meaning of this invention, when aligning a sequence to be examined (“query sequence”) with a reference sequence (“subject sequence”), the subject sequence must be represented at least 90% over the length of the individual alignment (“sequence coverage” at least 90%); alignments with a lower sequence coverage of the subject sequence are excluded for the determination of sequence identity for the purposes of this application. However, the query sequence may be longer than the length of the alignment, and the sequences represented in the alignment of the query sequence may be above or below 90%.

Preferably the nucleoside monophosphate kinase is employed in a concentration of at least 0.05 mU/ml; preferably of at least 0.1 mU/ml; preferably of at least 0.2 mU/ml; preferably of at least 0.3 mU/ml; preferably of at least 0.4 mU/ml; preferably of at least 0.5 mU/ml; preferably of at least 0.6; preferably of at least 0.7 mU/ml; preferably of at least 0.8 mU/ml; preferably of at least 0.9 mU/ml; more preferably of at least 1.0 mU/ml.

In step (c) of the process according to the invention, a saccharide donor is provided.

For the purpose of the specification a saccharide donor is to be understood as an organic or inorganic compound capable of donating a saccharide moiety i.e. a molecule comprising a saccharide moiety bound to a leaving group. Leaving groups are to be preferably regarded as molecular fragments which deliver said saccharide moiety upon displacement and can be of any of the type know to the person skilled in the art such anion cations or neutral molecules.

Preferably, the saccharide donor is a, disaccharide, oligosaccharide, or polysaccharide.

Preferably, the saccharide donor is or comprises a moiety derived from galactose, glucose, fucose, mannose, glucuronic acid, sialyic acid, N-acetylgalactosamine, N-acetylglucosamin, tagatose, talose, xylose, arabinose, rhamnose, starch, or inulin.

Preferably, the saccharide donor is or comprises a moiety derived from galactose and/or glucose; preferably sucrose.

Preferably the saccharide donor is added to the reaction in a concentration range of from 100 mM up to 2000 mM, or 200 mM up to 2000 mM, or 300 mM up to 2000 mM, or 400 mM up to 2000 mM, or 500 mM up to 2000 mM, or 600 mM up to 2000 mM, or 700 mM up to 2000 mM, or 800 mM up to 2000 mM, or 900 mM up to 2000 mM, or 1000 mM up to 2000 mM, or 1100 mM up to 2000 mM, or 1200 mM up to 2000 mM, or 1300 mM up to 2000 mM, or 1400 mM up to 2000 mM, or 1500 mM up to 2000 mM, or 1600 mM up to 2000 mM, or 1700 mM up to 2000 mM, or 1800 mM up to 2000 mM, or 1900 mM up to 2000 mM, or preferably in the range of 500 mM up to 1100 mM, or 600 mM up to 1100 mM, or 700 mM up to 1100 mM.

In step (d) of the process according to the invention, the nucleoside diphosphate obtained in step (b) is reacted with the saccharide of the saccharide donor provided in step (c) under catalysis of the first glycosyl transferase thereby obtaining a nucleoside diphosphate saccharide.

In one preferred embodiment, the first glycosyl transferase is a sucrose synthase belonging to EC class 2.4.1.13.

For the purpose of the specification, a sucrose synthase preferably is a glycosyltransferase that reversibly catalyzes the chemical reaction of NDP-glucose and D-fructose to NDP and sucrose.

Preferably, said sucrose synthase is a uridine diphosphate specific sucrose synthase.

In preferred embodiments, the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a wild-type polypeptide sequence of Arabidopsis thaliana; preferably a wild-type polypeptide sequence of Arabidopsis thaliana corresponding to SEQ ID NO: 9.

Preferably, the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2.

Preferably the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3.

The first glycosyl transferase according to the invention comprises amino acid sequences with a defined identity to the amino acid sequence of SEQ ID NO: 2 or SEQ ID NO: 3. This means that the first glycosyl transferase according to the invention may comprise said amino acid sequence as a subsequence of its overall amino acid sequence, or that the first glycosyl transferase according to the invention may essentially consist of said amino acid sequence. When the first glycosyl transferase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the first glycosyl transferase is to be immobilized on a solid support, e.g. for purification purposes.

Preferably, first glycosyl transferase is employed is employed in a concentration of at least 5 mU/ml; preferably of at least 10 mU/ml; preferably of at least 20 mU/ml; preferably of at least 25 mU/ml; preferably of at least 30 mU/ml; preferably of at least 35 mU/ml; preferably of at least 40 mU/ml; preferably of at least 45 mU/ml; more preferably of at least 50 mU/ml.

In step (e) of the process according to the invention, an organic compound having a nucleophilic group is provided.

Preferably, the organic compound having a nucleophilic group is selected from the group consisting of terpenes, steroids, carotenoids, peptides, proteins, antibodies, sweeteners, steviol glycosides, rebaudiosides, polyphenols, oligosaccharides, and polysaccharides.

For the purpose of the specification a nucleophilic group is preferably as a functional substituent of an organic compound characterized by the presence of electron-rich atoms which can be donated electron-poorer acceptors such as electrophiles for the purpose of forming a new bond in the course of a reaction. Thus the nucleophilic group of said organic compound is preferably of the type R-XHy wherein X preferably corresponds to an atom belonging to the 15^{th}, 16^{th}, or 17^{th} group of the periodic table, and y is an integer number equal or greater than one. Likewise saturated or unsaturated, unsubstituted or mono- or polysubstituted, branched or unbranched homo- or heteroaliphatic or linear or cyclic aliphatic or aromatic, organic or inorganic substituents presenting a free lone pair as such or following deprotonation are to be comprised within the meaning of the present disclosure as suitable nucleophilic groups.

Example of preferred nucleophilic groups are selected from the group comprising OH, NH₂, SH, PH or corresponding anions.

In a preferred embodiment the nucleophilic group is OH or an anion deriving by its deprotonation.

Preferably, the organic compound having a nucleophilic group is selected from the group consisting of terpenes, steroids, carotenoids, peptides, proteins, antibodies, sweeteners, steviol glycosides, rebaudiosides, polyphenols, oligosaccharides, and polysaccharides.

Preferably, the organic compound having a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside I2, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2020112957A1 which is hereby incorporated by reference, synthetic steviol glycosides, and combinations thereof.

Preferably, the organic compound having a nucleophilic group is a rebaudioside; more preferably rebaudioside A.

Preferably, the organic compound having a nucleophilic group is a polyphenol; preferably a mono or polysubstituted stilbenoide glucoside; more preferably polydatin.

Preferably, the organic compound having a nucleophilic group is an oligosaccharide; preferably an oligo-saccharide containing three saccharide units; more preferably, Lacto-N-triose II.

Preferably, the organic compound having a nucleophilic group is employed a concentration of at least 1 mM; preferably at least 1.5 mM; preferably at least 2.0 mM.

Preferably, the organic compound having a nucleophilic group is employed a concentration of at most 500 mM; preferably at most 100 mM more preferably at most 10 mM.

In a preferred embodiment the organic compound having a nucleophilic group is employed a concentration within the range of from 1 to 500 mM, or 2 mM to 500 mM, or 3 mM to 500 mM, or 4 mM to 500 mM, or 5 mM to 500 mM, or 6 mM to 500 mM, or 7 mM to 500 mM, or 8 mM to 500 mM, or 9 mM to 500 mM; preferably from 10 mM to 500 mM; more preferably from 40 mM to 500 mM, or 50 mM to 500 mM, or 60 mM to 100 mM, or 70 mM to 500 mM, or 80 mM to 500 mM, or 90 mM to 500; and more preferably from 100 to 500 mM.

In step (f) of the process according to the invention, the nucleoside diphosphate saccharide obtained in step (d) is reacted with the organic compound having a nucleophilic group provided in step (e) under catalysis of at least the second glycosyltransferase thereby obtaining the glycosylated organic compound.

Preferably, the second glycosyltransferase is a nucleotide sugar-dependent glycosyltransferase.

Preferably, the second glycosyltransferase is selected from the group consisting of uridine diphosphate glycosyltransferases, adenosine diphosphate glycosyltransferases, cytidine diphosphate glycosyltransferase, guanosine diphosphate glycosyltransferase, thymidine diphosphate glycosyltransferase; preferably an uridine diphosphate dependent glycosyltransferase.

Preferably, the second glycosyltransferase is capable of catalyzing the transfer of a sugar moiety from a uridine diphosphate sugar onto the nucleophilic group of the organic compound.

Preferably, the second glycosyl transferase is a uridine diphosphate dependent glycosyltransferase belonging to EC class 2.4.1X.

Preferably, the second glycosyltransferase is capable of catalyzing the transfer of a sugar moiety onto one or more position of an organic compound having a nucleophilic group wherein the organic compound presenting a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside I2, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2020112957A1 which is hereby incorporated by reference, synthetic steviol glycosides, and combinations thereof.

Preferably, the second glycosyl transferase is a uridine diphosphate dependent glycosyltransferase belonging to EC class 2.4.1X.

In a preferred embodiment the second glycosyl transferase is a uridine diphosphate dependent glycosyltransferase comprising a primary sequence selected from the listing of GenInfo identifier numbers presented in the following list (for the purposes of the specification also referred to as “List 1”):

| | | | | | | |
|---|---|---|---|---|---|---|
| 397567 | 30680413 | 115480946 | 147798902 | 218193594 | 225443294 | 454245 |
| 32816174 | 116310259 | 147811764 | 218193942 | 225444853 | 1359905 | 32816178 |
| 116310985 | 147827151 | 219885307 | 225449296 | 1685003 | 34393978 | 116788066 |
| 147836230 | 222615927 | 225449700 | 1685005 | 37993665 | 116788606 | 147839909 |
| 222619587 | 225454338 | 2191136 | 37993671 | 116789315 | 147846163 | 222623142 |
| 225454340 | 2501497 | 37993675 | 119394507 | 147855977 | 222625633 | 225454342 |
| 2911049 | 39104603 | 119640480 | 148905778 | 222625635 | 225454473 | 4218003 |
| 41469414 | 122209731 | 148905999 | 222636620 | 225454475 | 4314356 | 41469452 |
| 125526997 | 148906835 | 222636621 | 225458362 | 13492674 | 42566366 | 125534279 |
| 148907340 | 222636628 | 225461551 | 13492676 | 42570280 | 125534461 | 148908935 |
| 222636629 | 225461556 | 15217773 | 42572855 | 125540090 | 148909182 | 224053242 |
| 225461558 | 15217796 | 44890129 | 125541516 | 148909920 | 224053386 | 225469538 |
| 15223396 | 46806235 | 125545408 | 148910082 | 224055535 | 225469540 | 15223589 |
| 50284482 | 125547340 | 148910154 | 224056138 | 226316457 | 15227766 | 51090402 |
| 125547520 | 148910612 | 224056160 | 226492603 | 15230017 | 51090594 | 125554547 |
| 148910769 | 224067918 | 226494221 | 15231757 | 52839682 | 125557592 | 156138791 |
| 224072747 | 226495389 | 15234056 | 56550539 | 125557593 | 156138797 | 224080189 |
| 226495945 | 15234195 | 62734263 | 125557608 | 156138799 | 224091845 | 226502400 |
| 15234196 | 62857204 | 125559566 | 156138803 | 224094703 | 226507980 | 15238503 |
| 62857206 | 125563266 | 165972256 | 224100653 | 226531147 | 15239523 | 62857210 |
| 125571055 | 168016721 | 224100657 | 226532094 | 15239525 | 62857212 | 125579728 |
| 171674071 | 224101569 | 238477377 | 15239543 | 75265643 | 125588307 | 171906258 |
| 224103105 | 240254512 | 15239937 | 75285934 | 125589492 | 183013901 | 224103633 |
| 242032615 | 15240305 | 75288884 | 125599469 | 183013903 | 224103637 | 242032621 |
| 15240534 | 77550661 | 125601477 | 186478321 | 224109218 | 242038423 | 15982889 |
| 77556148 | 126635837 | 187373030 | 224114583 | 242043290 | 18086351 | 82791223 |
| 126635845 | 187373042 | 224116284 | 242044836 | 18418378 | 83778990 | 126635847 |
| 190692175 | 224120552 | 242051252 | 18418380 | 89953335 | 126635863 | 194701936 |
| 224121288 | 242056217 | 18418382 | 110741436 | 126635867 | 195620060 | 224121296 |
| 242056219 | 19743740 | 110743955 | 126635883 | 209954691 | 224121300 | 242056663 |
| 19911201 | 115438196 | 126635887 | 209954719 | 224130358 | 242059339 | 20149064 |
| 115438785 | 133874210 | 209954725 | 224140703 | 242059341 | 20260654 | 115441237 |
| 133874212 | 209954733 | 224143404 | 242060922 | 21435782 | 115454819 | 145358033 |
| 210063105 | 224143406 | 242067411 | 21553613 | 115456047 | 147772508 | 210063107 |
| 224144306 | 242067413 | 21593514 | 115457492 | 147776893 | 212275846 | 224285244 |
| 242076258 | 22759895 | 115459312 | 147776894 | 216296854 | 225431707 | 242076396 |
| 23955910 | 115464719 | 147776895 | 217074506 | 225435532 | 242084750 | 26452040 |
| 115471069 | 147786916 | 218185693 | 225436321 | 242091005 | 28393204 | 115471071 |
| 147798900 | 218187075 | 225440041 | 242095206 | 30679796 | 115474009 | 147798901 |
| 218189427 | 225441116 | 242345159 | 242345161 | 297724601 | 326492035 | 356523945 |
| 357140904 | 359486938 | 255536859 | 297725463 | 326493430 | 356523957 | 357165849 |
| 359487055 | 255538228 | 297728331 | 326500410 | 356523959 | 357165852 | 359488135 |
| 255541676 | 297738632 | 326506816 | 356523961 | 357168415 | 359488708 | 255547075 |
| 297745347 | 326507826 | 356523963 | 357437837 | 359493630 | 255552620 | 297745348 |
| 326508394 | 356524387 | 357442755 | 359493632 | 255552622 | 297795735 | 326509445 |
| 356524403 | 357442757 | 359493634 | 255555343 | 297796253 | 326511261 | 356527181 |
| 357445729 | 359493636 | 255555361 | 297796257 | 326511866 | 356533209 | 357445731 |
| 359493815 | 255555363 | 297796261 | 326512412 | 356533852 | 357445733 | 359495856 |
| 255555365 | 297797587 | 326517673 | 356534718 | 357446799 | 359495858 | 255555369 |
| 297798502 | 326518800 | 356535480 | 357446805 | 359495869 | 255555373 | 297799226 |
| 326521124 | 356542996 | 357452779 | 359495871 | 255555377 | 297805988 | 326525567 |
| 356543136 | 357452781 | 359497638 | 255556812 | 297807499 | 326525957 | 356543932 |
| 357452783 | 359807261 | 255556818 | 297809125 | 326526607 | 356549841 | 357452787 |
| 374256637 | 255563008 | 297809127 | 326527141 | 356549843 | 357452789 | 377655465 |
| 255564074 | 297811403 | 326530093 | 356554358 | 357452791 | 378405177 | 255564531 |
| 297820040 | 326534036 | 356554360 | 357452797 | 378829085 | 255572878 | 297821483 |
| 326534312 | 356558606 | 357452799 | 387135070 | 255577901 | 297825217 | 332071132 |
| 356560333 | 357470367 | 387135072 | 255583249 | 297832276 | 339715876 | 356560599 |
| 357472193 | 387135078 | 255583253 | 297832280 | 342306012 | 356560749 | 357472195 |
| 387135092 | 255583255 | 297832518 | 342306016 | 356566018 | 357474295 | 387135094 |
| 255585664 | 297832520 | 343457675 | 356566169 | 357474493 | 387135098 | 255585666 |
| 297840825 | 343457677 | 356566173 | 357474497 | 387135100 | 255634688 | 297840827 |
| 350534960 | 356567761 | 357474499 | 387135134 | 255644801 | 297847402 | 356498085 |
| 356574704 | 357490035 | 387135136 | 255645821 | 297849372 | 356499771 | 356576401 |
| 357493567 | 387135174 | 255647456 | 300078590 | 356499777 | 356577660 | 357497139 |
| 387135176 | 255648275 | 300669727 | 356499779 | 357114993 | 357497581 | 387135184 |
| 260279126 | 302142947 | 356501328 | 357115447 | 357497671 | 387135186 | 260279128 |
| 302142948 | 356502523 | 357115451 | 357500579 | 387135188 | 261343326 | 302142950 |
| 356503180 | 357115453 | 357504663 | 387135190 | 283132367 | 302142951 | 356503184 |
| 357116080 | 357504691 | 387135192 | 283362112 | 302765302 | 356503295 | 357116928 |
| 357504699 | 387135194 | 289188052 | 302796334 | 356504436 | 357117461 | 357504707 |
| 387135282 | 295841350 | 302811470 | 356504523 | 357117463 | 357505859 | 387135284 |
| 296088529 | 302821107 | 356504765 | 357117829 | 357510851 | 387135294 | 296090415 |
| 302821679 | 356511113 | 357117839 | 357516975 | 387135298 | 296090524 | 319759260 |
| 356515120 | 357125059 | 359477003 | 387135300 | 296090526 | 319759266 | 356517088 |
| 357126015 | 359477998 | 387135302 | 297599503 | 320148814 | 356520732 | 357134488 |
| 359478043 | 387135304 | 297601531 | 326489963 | 356522586 | 357135657 | 359478286 |
| 387135312 | 297611791 | 326490273 | 356522588 | 357138503 | 359484299 | 387135314 |
| 297722841 | 326491131 | 356522590 | 357139683 | 359486936 | 387135316 | 387135318 |
| 449440433 | 460376293 | 460413408 | 462423864 | 475546199 | 387135320 | 449445896 |
| 460378310 | 460416351 | 470101924 | 475556485 | 387135322 | 449446454 | 460380744 |
| 462394387 | 470102280 | 475559699 | 387135324 | 449447657 | 460381726 | 462394433 |
| 470102858 | 475578293 | 387135326 | 449449002 | 460382093 | 462394557 | 470104211 |
| 475591753 | 387135328 | 449449004 | 460382095 | 462395646 | 470104264 | 475593742 |
| 388493506 | 449449006 | 460382754 | 462395678 | 470104266 | 475612072 | 388495496 |
| 449451379 | 460384935 | 462396388 | 470106317 | 475622476 | 388498446 | 449451589 |
| 460384937 | 462396389 | 470106357 | 475622507 | 388499220 | 449451591 | 460385076 |
| 462396419 | 470115448 | 475623787 | 388502176 | 449451593 | 460385872 | 462396542 |
| 470130404 | 482550481 | 388517521 | 449453712 | 460386018 | 462397507 | 470131550 |
| 482550499 | 388519407 | 449453714 | 460389217 | 462399998 | 470136482 | 482550740 |
| 388521413 | 449453716 | 460394872 | 462400798 | 470136484 | 482550999 | 388827901 |
| 449453732 | 460396139 | 462401217 | 470136488 | 482552352 | 388827903 | 449457075 |
| 460397862 | 462402118 | 470136492 | 482554970 | 388827907 | 449467555 | 460397864 |
| 462402237 | 470137933 | 482555336 | 388827909 | 449468742 | 460398541 | 462402284 |
| 470137937 | 482555478 | 388827913 | 449495638 | 460403139 | 462402416 | 470140422 |
| 482556454 | 393887637 | 449495736 | 460403141 | 462404228 | 470140426 | 482557289 |
| 393887646 | 449499880 | 460403143 | 462406358 | 470140908 | 482558462 | 393887649 |
| 449502786 | 460403145 | 462408262 | 470141232 | 482558508 | 393990627 | 449503471 |
| 460405998 | 462409325 | 470142008 | 482558547 | 397746860 | 449503473 | 460407578 |
| 462409359 | 470142010 | 482561055 | 397789318 | 449515857 | 460407590 | 462409777 |
| 470142012 | 482561555 | 413924864 | 449518643 | 460409128 | 462411467 | 470143607 |
| 482562795 | 414590349 | 449519559 | 460409134 | 462414311 | 470143939 | 482562850 |
| 414590661 | 449522783 | 460409136 | 462414416 | 470145404 | 482565074 | 414591157 |
| 449524530 | 460409459 | 462414476 | 473923244 | 482566269 | 414879558 | 449524591 |
| 460409461 | 462415526 | 474114354 | 482566296 | 414879559 | 449528823 | 460409463 |
| 462415603 | 474143634 | 482566307 | 414879560 | 449528825 | 460409465 | 462415731 |
| 474202268 | 482568689 | 414888074 | 449534021 | 460409467 | 462416307 | 474299266 |
| 482570049 | 431812559 | 460365546 | 460410124 | 462416920 | 474363119 | 482570572 |
| 449432064 | 460366882 | 460410126 | 462416922 | 474366157 | 482575121 | 449432066 |
| 460369823 | 460410128 | 462416923 | 474429346 | 449433069 | 460369829 | 460410130 |
| 462416924 | 475432777 | 449436944 | 460369831 | 460410132 | 462417401 | 475473002 |
| 449438665 | 460369833 | 460410134 | 462419769 | 475489790 | 449438667 | 460370755 |
| 460410213 | 462420317 | 475511330 | 449440431 | 460374714 | 460411200 | 462423366 |
| 475516200 | | | | | | |

In other preferred embodiments the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to any of the polypeptide sequences of List1.

In preferred embodiments the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to a wild type polypeptide sequence of Solanum lycopersicum; preferably a wild-type polypeptide sequence of Solanum lycopersicum having NCBI accession number XP_004250485.1.

In other preferred embodiments the second glycosyl transferase is a uridine diphosphate dependent glycosyltransferase comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4.

Preferably, the second glycosyltransferase is capable of catalyzing glycosylation of a polyphenol; preferably a mono di- or polysubstituted stilbenoide glucoside; more preferably polydatin.

In preferred embodiments the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5.

Preferably, the second glycosyltransferase is capable of catalyzing glycosylation of oligosaccharide preferably an oligosaccharide containing three saccharide units; more preferably, Lacto-N-triose II.

Preferably, the second glycosyl transferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%; or preferably at least 93%, or at least 94%, or at least 95%, or at least 96%, or at least 97%; and in particular at least 98%, or at least 99%, or 100%, in each case to SEQ ID NO: 6.

Preferably, the second glycosyl transferase according to the invention comprises such an amino acid sequence with a defined identity to the amino acid sequences of SEQ ID NO: 4, SEQ ID NO: 5 or SEQ ID NO: 6. This means that the second glycosyl transferase according to the invention may comprise said amino acid sequence as a subsequence of its overall amino acid sequence, or that the first glycosyl transferase according to the invention may essentially consist of said amino acid sequence. When the second glycosyl transferase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous.

Preferably, the second glycosyltransferase is employed in a concentration of at least 0.2 mU/ml; preferably of at least 0.4 mU/ml; preferably of at least 0.6 mU/ml; preferably of at least 0.8 mU/ml; preferably of at least 1.0 mU/ml; preferably of at least 1.2 mU/ml; preferably of at least 1.4 mU/ml; preferably of at least 1.6 mU/ml; preferably of at least 1.8 mU/ml; preferably of at least 2.0 mU/ml; preferably of at least 2.2 mU/ml; preferably of at least 2.4 mU/ml; preferably of at least 2.6 mU/ml; preferably of at least 2.8 mU/ml; preferably of at least 3.0 mU/ml; preferably of at least 3.2 mU/ml; preferably of at least 3.3 mU/ml.

In other preferred embodiments, step (f) involves the use of a third glycosyltransferase in addition to the second glycosyltransferase

Preferably, the third glycosyltransferase is selected from the group consisting of uridine diphosphate glycosyltransferases, adenosine diphosphate glycosyl-transferases, cytidine diphosphate glycosyltransferase, guanosine diphosphate glycosyltransferase, thymidine diphosphate glycosyltransferase; preferably an uridine diphosphate dependent glycosyltransferase.

In preferred embodiments the third glycosyltransferase is capable of catalyzing the transfer of a sugar moiety onto one or more position of an organic compound having a nucleophilic group wherein the organic compound presenting a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside I2, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3,rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2020112957A1 which is hereby incorporated by reference, synthetic steviol glycosides, and combinations thereof.

Preferably, the third glycosyl transferase is a uridine diphosphate dependent glycosyltransferase belonging to EC class 2.4.1X.

Preferably, the third glycosyl transferase is a uridine diphosphate dependent glycosyltransferase comprising a primary sequence selected from the listing of GenInfo of List 1 as described above.

In other preferred embodiments the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to any of the polypeptide sequence of List 1 as described above.

Preferably, the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%; or preferably at least 93%, or at least 94%, or at least 95%, or at least 96%, or at least 97%; and in particular at least 98%, or at least 99%, or 100%, in each case to a wild-type polypeptide sequence of Stevia rebaudiana; preferably a wild-type polypeptide sequence of Stevia rebaudiana having GenBank number AAR06912.1.

In preferred embodiments the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7.

Preferably, the third glycoside transferase according to the invention comprises such an amino acid sequence with a defined identity to the amino acid sequence of SEQ ID NO: 7. This means that the third glycoside transferase according to the invention may comprise said amino acid sequence as a subsequence of its overall amino acid sequence, or that the third glycosyl transferase according to the invention may essentially consist of said amino acid sequence. When the third glycoside transferase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the third glycosyl transferase is to be immobilized on a solid support, e.g. for purification purposes.

Preferably, the third glycosyltransferase is employed in a concentration of at least 1.0 mU/ml; preferably of at least 2.0 mU/ml; preferably of at least 3.0 mU/ml; preferably of at least 4.0 mU/ml; preferably of at least 5.0 mU/ml; preferably of at least 6.0 mU/ml; preferably of at least 7.0 mU/ml; preferably of at least 8.0 mU/ml; preferably of at least 9.0 mU/ml; preferably of at least 10 mU/ml; preferably of at least 11 mU/ml; preferably of at least 12 mU/ml; preferably of at least 13 mU/ml; preferably of at least 14 mU/ml; preferably of at least 15 mU/ml; preferably of at least 16 mU/ml.

In preferred embodiments of the process according to the invention the nucleoside monophosphate kinase, the first glycosyltransferase, and the second glycosyltransferase are the only enzymes that are employed in the process.

In other preferred embodiments, the nucleoside diphosphate saccharide obtained in step (d) comprises a sugar moiety and wherein the process comprises the further step of (h) converting the sugar moiety into an epimer thereof under catalysis of an epimerase.

For the purpose of the specification, an epimerase is preferably as an enzyme able to catalyze the conversion of a molecule from one isomer (epimer) to another (epimer).

Preferably, the sugar moiety is a glucose moiety that is converted into a galactose moiety, and wherein the epimerase is a glucose galactose epimerase; preferably a UDP-glucose 4-epimerase.

Preferably, the epimerase is UDP-glucose 4-epimerase belonging to EC class EC 5.1.3.2.

Preferably, the epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8.

Preferably, the epimerase according to the invention comprises such an amino acid sequence with a defined identity to the amino acid sequence of SEQ ID NO: 8. This means that the epimerase according to the invention may comprise said amino acid sequence as a subsequence of its overall amino acid sequence, or that the epimerase according to the invention may essentially consist of said amino acid sequence. When the epimerase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the epimerase is to be immobilized on a solid support, e.g. for purification purposes.

In preferred embodiments of the process according to the invention, the phosphate donor and the nucleoside monophosphate are selected independently of one another and are employed in a total concentration of at least 0.05 mM; preferably at least 0.1 mM; more preferably at least 0.2 mM.

For the purpose of the total concentration preferably is the sum of the molar concentration of each constituent.

Preferably, the phosphate donor and the nucleoside monophosphate are employed in a total concentration of at most 8.0 mM; preferably at most 4.0 mM.

Preferably, the phosphate donor and the nucleoside monophosphate are employed in a total concentration within the range of 0.55±0.5 mM, or 0.6±0.5 mM, or 0.8±0.5 mM, or 1.0±0.5 mM, or 1.2±0.5 mM, or 1.4±0.5 mM, or 1.6±0.5 mM, or 1.8±0.5 mM, or 2.0±0.5 mM, or 2.2±0.5 mM, or 2.4±0.5 mM, or 2.6±0.5 mM, or 2.8±0.5 mM, or 3.0±0.5 mM, or 3.2±0.5 mM, or 3.4±0.5 mM, or 3.6±0.5 mM, or 3.8±0.5 mM, or 4.0±0.5 mM, or 4.2±0.5 mM, or 4.4±0.5 mM, or 4.6±0.5 mM, or 4.8±0.5 mM, 5.0±0.5 mM, or 5.2±0.5 mM, or 5.4±0.5 mM, or 5.6±0.5 mM, or 5.8±0.5 mM, or 6.0±0.5 mM, 6.2±0.5 mM, or 6.4±0.5 mM, or 6.6±0.5 mM, or 6.8±0.5 mM, or 7.0±0.5 mM, or 7.2±0.5 mM; 7.4±0.5 mM, or 7.5±0.5 mM; preferably within the range of 0.1±0.4 mM, or 0.6±0.4 mM, or 0.8±0.4 mM, or 1.0±0.4 mM, or 1.2±0.4 mM, or 1.4±0.4 mM, or 1.6±0.4 mM, or 1.8±0.4 mM, or 2.0±0.4 mM, or 2.2±0.4 mM, or 2.4±0.4 mM, or 2.6±0.4 mM, or 2.8±0.4 mM, or 3.0±0.4 mM, or 3.2±0.4 mM, or 3.4±0.4 mM, or 3.6±0.4 mM, or 3.8±0.4 mM, or 4.0±0.4 mM, or 4.2±0.4 mM, or 4.4±0.4 mM, or 4.6±0.4 mM, or 4.8±0.4 mM, 5.0±0.4 mM, or 5.2±0.4 mM, or 5.4±0.4 mM, or 5.6±0.4 mM, or 5.8±0.4 mM, or 6.0±0.4 mM, 6.2±0.4 mM, or 6.4±0.4 mM, or 6.6±0.4 mM, or 6.8±0.4 mM, or 7.0±0.4 mM, or 7.2±0.4 mM; 7.4±0.4 mM, or 7.5±0.4 mM; more preferably within the range of 0.1±0.3 mM, or 0.6±0.3 mM, or 0.8±0.3 mM, or 1.0±0.3 mM, or 1.2±0.3 mM, or 1.4±0.3 mM, or 1.6±0.3 mM, or 1.8±0.3 mM, or 2.0±0.3 mM, or 2.2±0.3 mM, or 2.4±0.3 mM, or 2.6±0.3 mM, or 2.8±0.3 mM, or 3.0±0.3 mM, or 3.2±0.3 mM, or 3.4±0.3 mM, or 3.6±0.3 mM, or 3.8±0.3 mM, or 4.0±0.3 mM, or 4.2±0.3 mM, or 4.4±0.3 mM, or 4.6±0.3 mM, or 4.8±0.3 mM, 5.0±0.3 mM, or 5.2±0.3 mM, or 5.4±0.3 mM, or 5.6±0.3 mM, or 5.8±0.3 mM, or 6.0±0.3 mM, 6.2±0.3 mM, or 6.4±0.3 mM, or 6.6±0.3 mM, or 6.8±0.3 mM, or 7.0±0.3 mM, or 7.2±0.3 mM; 7.4±0.3 mM, or 7.5±0.3 mM.

It has been further surprisingly found that employment of specific molar ratios between the nucleoside monophosphate and the phosphate donor allows for achieving a relative conversion of the glycosylated organic compound close or higher than that which would otherwise be achieved in a reaction relying on regeneration of the phosphate donor.

For the purpose of the present disclosure, the relative conversion is the percent conversion compared to a reference reaction. In that regard, conversion should be preferably be interpreted as final molar amount of product achieved after the reaction took place, compared to the sum of the molar amount of product and educts in percent.

Preferably, the nucleoside monophosphate and the phosphate donor are employed in a molar ratio of at least 0.05; preferably at least 0.3; more preferably at least 0.65.

Preferably, the nucleoside monophosphate and the phosphate donor are employed in a molar ratio of at most 8.0.

Preferably, the nucleoside monophosphate and the phosphate donor are employed in a molar ratio within the range 0.05 to 8.0, or 0.1 to 8.0 or 0.8±0.5 to 8.0, or 1.0 to 8.0, or 1.2 to 8.0, or 1.4 to 8.0, or 1.6 to 8.0, or 1.8 to 8.0, or 2.0 to 8.0, or 2.2 to 8.0, or 2.4 to 8.0, or 2.6 to 8.0, or 2.8 to 8.0, or 3.0 to 8.0, or 3.2 to 8.0, or 3.4 to 8.0, or 3.6 to 8.0, or 3.8 to 8.0, or 4.0 to 8.0, or 4.2 to 8.0, or 4.4 to 8.0, or 4.6 to 8.0, or 4.8, 5.0 to 8.0, or 5.2 to 8.0, or 5.4 to 8.0, or 5.6 to 8.0, or 5.8 to 8.0, or 6.0, 6.2 to 8.0, or 6.4 to 8.0, or 6.6 to 8.0, or 6.8 to 8.0, or 7.0 to 8.0 preferably within the range of 0.6 to 4.5, or 0.8 to 4.5, or 1.0 to 4.5, or 1.2 to 4.5, or 1.4 to 4.5, or 1.6 to 4.5, or 1.8 to 4.5, or 2.0 to 4.5, or 2.2 to 4.5, or 2.4 to 4.5, or 2.6 to 4.5, or 2.8 to 4.5, or 3.0 to 4.5 more preferably within the range or 0.6 to 1.5, or 0.8 to 1.5, or 1.0 to 1.5, or 1.2 to 1.5, or 1.4 to 1.5, or 1.6 to 1.5, or 1.8 to 1.5 and even more preferably within the range 0.6 to 1.5 such as 0.6±0.5, or 0.8±0.5, or 1.0±0.5, or 1.2±0.5, or 1.4±0.5.

Preferably, the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0.

Preferably, the process is carried out at a pH of at least 2.5; preferably at least 4.5; more preferably of at least 6.5.

Preferably, the process is carried out at a pH of at most 14; preferably at most 9.5; more preferably of at most 7.5.

Preferably, the process is carried out at a pH within the range of 3.75±1.25, or 5.75±1.25, or 6.75±1.25, or 7.75±1.25, or 8.75±1.25, or 9.75±1.25, or 10.75±1.25, or 11.75±1.25, or 12.75±1.25 preferably within the range of 3.75±1.15, or 5.75±1.15, or 6.75±1.15, or 7.75±1.15, or 8.75±1.15, or 9.75±1.15, or 10.75±1.15, or 11.75±1.15, or 12.75±1.15; more preferably within the range of 3.75±1.05, or 5.75±1.05, or 6.75±1.05, or 7.75±1.05, or 8.75±1.05, or 9.75±1.05, or 10.75±1.05, or 11.75±1.05, or 12.75±1.05.

Preferably, wherein the process is carried out at a temperature of at least 20° C.; preferably at least 25° C.; more preferably of at least 30° C.

Preferably, the process is carried out at a temperature of at most 100° C., or at most 65° C.; preferably at most 55° C.; more preferably of at most 45° C.

Preferably, the process is carried out at a temperature within the range of 28±8° C., or 36±8° C., or 44±8° C., or 52±8° C., or 60±8° C., or 68±8° C., or 76±8° C., or 84±8° C.; 92±8° C. preferably within the range of 28±7° C., or 36±7° C., or 44±7° C., or 52±8° C., or 60±7° C., or 68±7° C., or 76±7° or 84±7° C.; 92±7° C.; more preferably within the range of 28±6° C., or 36±6° C., or 44±6° C., or 52±6° C., or 60±6° C., or 68±6° C., or 76±6° C., or 84±6° C.; 92±6° C.

In preferred embodiments, the process is carried out within a total reaction time comprised within the range of from 0.5 to 120 h; preferably from 24 to 120 h; more preferably from 35 to 120 h.

Preferably, the phosphate donor differs from the nucleoside monophosphate.

Preferably, the phosphate donor and the nucleoside monophosphate donor present different molecular weights.

Preferably, the phosphate donor and the nucleoside monophosphate differ by at least one carbon atom.

Preferably, the phosphate donor and the nucleoside monophosphate differ by at most one carbon atom.

Preferably, the phosphate donor differs from the nucleoside monophosphate in the nucleobase and/or in the number of the phosphate groups.

Preferably, the process is performed as a batch process.

Preferably, the process is performed as a continuous or as a semi-continuous process.

Preferably, at least one of steps (a), (b), (c), (d), (e) and (f) is repeated at least once.

Preferably, at least two of steps (a), (b), (c), (d), (e) and (f) are performed in a single reactor.

Preferably, all steps (a), (b), (c), (d), (e) and (f) are performed in a single reactor.

Preferably, steps (a), (c) and/or (e) are performed simultaneously.

Preferably, steps (b), (d) and/or (f) are performed simultaneously.

Preferably, the steps (a), (b), (c), (d), (e) and (f) are performed simultaneously.

In a preferred embodiment the process according to the invention comprises a step (g) of purifying the glycosylated organic compound.

Preferably, wherein step (g) involves a microfiltration step, an ion-exchange step, a crystallization step, or any combination of the foregoing.

Preferably, step (g) involves at least a crystallization step and wherein the crystallization step comprises dissolving the reaction mixture of step (f) in an appropriate first solvent and precipitating at least the glycosylated organic compound.

Preferably, the precipitation step involves lowering the temperature.

Preferably, the precipitation step involves addition of a second solvent wherein at least one component of the reaction mixture obtained in step (f) presents lower solubility than in said first solvent.

In preferred embodiments the process involves re-crystallization of the glycosylated organic compound by solvent evaporation.

In a preferred embodiment, the process according to the invention comprises the use of at least one enzyme as row cell extracts.

In other preferred embodiments of the process according to the invention either all or at least one enzyme is subjected to a purification step prior to being used.

Preferably, the at least one enzyme is the nucleoside monophosphate kinase

Preferably, the at least one enzyme is the first glycosyltransferase.

Preferably, the at least one enzyme is the second glycosyltransferase.

Preferably, the at least one enzyme is the epimerase.

Preferably, the at least one enzyme is the third glycosyltransferase.

In a preferred embodiment the process according to the invention involves the use of at least one enzyme that has been produced in genetically modified organism.

In this context, the at least one enzyme that has been produced in genetically modified organism is preferably the first glycosyltransferase according to the present disclosure as defined above.

Thus, in preferred embodiments, said enzyme that has been produced in genetically modified organism is a sucrose synthase, more preferably a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a wild-type polypeptide sequence of Arabidopsis thaliana; preferably a wild-type polypeptide sequence of Arabidopsis thaliana corresponding to SEQ ID NO: 9.

Preferably, said enzyme that has been produced in genetically modified organism is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2.

Preferably, said enzyme that has been produced in genetically modified organism is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3.

For the purpose of the specification, the genetically modified organism is preferably to be regarded an organism comprising a genetic modification relating to the insertion or deletion of one or more genes in an organism, said organism being selected from the group comprising but not limited to the organism listing presented in the following list (for the purpose of the specification also referred to as “List 2”):

| | | | |
|---|---|---|---|
| Bacillus atrophaeus | Bacillus natto | Bacillus subtilis | Bacillus subtilis 168 |
| Bacillus megaterium | Bacillus amyloliquefaciens | Bacillus sp. | Rhizobia sp. |
| Aspergillus fumigatus | Aspergillus oryzae | Escherichia sp. | Neurospora crassa |
| Pseudomonas aeruginosa | Escherichia coli | Saccharomyces cerevisiae | Photobacterium leiognathi |
| Pseudomonas sp. | Proteus vulgaris | Pseudomonas oleovorans | Salinivibrio costicola |
| Streptomyces avermitilis | Photobacterium phosphoreum | Trichoderma atroviride | Pseudomonas putida |
| Schizosaccharomyces pombe | Pseudomonas fluorescens | Streptomyces lividans | Trichoderma reesei |
| Saccharomyces boulardii | Streptomyces griseus | Candida utilis | Streptomyces sp. |
| Pichia pastoris | Saccharomyces carlsbergensis | Kluyveromyces lactis | Candida albicans |
| Phanerochaete chrysogenum | Hansenula polymorpha | Komagataella sp. | Yarrowia lipolytica |
| Enterococcus sp. | Komagataella pastoris | Zymomonas mobilis | Streptococcus sp. |
| Erwinia sp. | Aspergillus niger | Staphylococcus sp. | Rhizobium sp. |
| Shigella sp. | Listeria sp. | Azotobacter sp. | Serratia sp. |
| Klebsiella sp. | Clostridium sp. | Salmonella sp. | Paracoccus sp. |
| Corynebacterium glutamicum | Proteus sp. | Vitreoscilla sp. | |

For the purpose of the specification, a genetic modification is to be regarded as a genetic modification resulting in attenuation or elimination of the expression of one or more genes encoding for an enzyme having hydrolase activity by modification of promotor strength, by modification of activating or inhibitory sequences, by modification of ribosome binding sites, by introduction of frame shift mutations, by introduction of premature stop codons or preferably by complete or partial deletion the genes.

Preferably said hydrolase activity is a 5′-nucleotidase or UDP-sugar hydrolase activity.

In a preferred embodiment said enzyme having hydrolase activity belongs to EC 3.1.3.5 or EC 3.6.1.45 respectively.

Preferably said enzyme having hydrolase activity comprises a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a ushA gene product of Escherichia coli parental strain K-12 W3110 having GenBank number BAE76259.1

In a preferred embodiment of the invention, the genetically modified organism is Escherichia coli; preferably a genetically modified laboratory derivative of E. coli parental strain K-12 W3110.

The parental strain E. coli K-12 W3110 belongs to the well-defined taxonomic family of the Enterobacteriaceae. Said genetically modified laboratory derivative of E. coli parental strain K-12 W3110 has been created by site-directed recombination at different chromosomal loci to suit production purposes in terms of genetic stability, especially plasmid stability, and efficiency of expression and downstream enzymatic conversions. The expression of a number of proteases has been eliminated by deletion of the corresponding genes. Antibiotic-free selection of target clones has been enabled through deletion of one gene. One further gene has been deleted to prevent unwanted recombination effects. The gene coding for the T7 RNA polymerase from E. coli T7 phage and a second gene copy of lacI, a repressor naturally present in E. coli K-12 W3110, have been inserted into the genome of W3110 to achieve a strong and regulated enzyme expression. Optionally, the laboratory derivative of E. coli parental strain K-12 W3110 may have been further engineered to improve strain stability, expression yields or strain cultivation and growth,

Preferably said E. coli parental strain K-12 W3110 or its laboratory derivative comprises a genetic modification involving deletion of one or more genes encoding for an enzyme having hydrolase activity; preferably a 5′-nucleotidase or UDP-sugar hydrolase activity.

In a preferred embodiment said E. coli parental strain K-12 W3110 or its laboratory derivative comprises a genetic modification involving deletion of one or more genes encoding for an enzyme having hydrolase activity, said enzyme having hydrolase activity belonging to EC 3.1.3.5 or EC 3.6.1.45 respectively.

In a preferred embodiment said E. coli parental strain K-12 W3110 or its laboratory derivative comprises a genetic modification involving deletion of one or more genes encoding for an enzyme having hydrolase activity said enzyme having hydrolase activity comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a protein sequence coded by ushA from Escherichia coli parental strain K-12 W3110 having GenBank number BAE76259.1

Preferably said genetically E. coli parental strain K-12 W3110 or its laboratory derivative comprises a genetic modification resulting in attenuation or elimination of the expression of ushA by modification of promotor strength, by modification of activating or inhibitory sequences, by modification of the ribosome binding site, by introduction of frame shift mutations, by introduction of premature stop codons or preferably by complete or partial deletion of the gene ushA.

In this regard, following genetic modification by deletion of the gene, preferably of the gene ushA, enzymes can be produced by incorporation of the genetic information into the cell. The genetic information may preferably be provided e.g. by a plasmid based expression construct carrying the enzyme coding gene or by insertion of the enzyme coding gene into genomic DNA. The expression of the gene may preferably be controlled by a promoter element adjacent to the enzyme coding gene. The enzyme is then produced during cultivation of the cell under conditions suitable to enable the expression of the enzyme coding gene. The so obtained generically modified enzyme is then preferably subjected to post processing steps suitable to render it for in vitro application. This could include the disruption of the cells, the separation of cell debris, the separation of small molecular metabolites, any method for enrichment of the desired enzymatic activity e.g. by chromatographic methods, precipitation, filtration etc. or by formulation to improve stability during storage or application.

For the latter purpose, any known genetic modification techniques can be used which are known to the person skilled in the art (see for example D K Baranwal et. al. (2013) “Gene knockout technology and its application”, Biologix, II(I), 55-59, which is hereby incorporated by reference).

In a preferred embodiment, the process according to the invention does not, in step (b), comprise regeneration of the phosphate donor.

In other preferred embodiments of the process according to the invention, a reaction product of the phosphate donor is obtained as a byproduct in step (b) besides the nucleoside diphosphate, whereby the process comprises the further steps of
(i) regenerating the reaction product of the phosphate donor thereby obtaining regenerated phosphate donor; and
(j) optionally, recirculating at least a portion of the regenerated phosphate donor to step (b).

In other preferred embodiments of the process according to the invention, a reaction product of the nucleoside diphosphate saccharide is obtained as a byproduct in step (f) besides the glycosylated organic compound, whereby the process comprises the further steps of
(k) regenerating the reaction product of the nucleoside diphosphate saccharide thereby obtaining regenerated nucleoside monophosphate, regenerated nucleoside diphosphate, or regenerated nucleoside diphosphate saccharide; and
(l) optionally, recirculating at least a portion of the regenerated nucleoside monophosphate to step (b), or at least a portion of the regenerated nucleoside diphosphate to step (d), or at least a portion of the regenerated nucleoside diphosphate saccharide to step (f), respectively.

For the purpose of the specification, the regeneration of step (i) or (k) is to be preferably intended as a process step by which the reaction product of the phosphate donor produced in step (b) and/or reaction product of the nucleoside diphosphate saccharide produced in step (f) is subjected to an enzymatic or chemical conversion aimed at reconstituting the original physical of the corresponding precursors. Said regeneration might take place in a continuous setting or optionally, may involve a prior step in which the reaction product of the phosphate donor produced in step (b) and/or reaction product of the nucleoside diphosphate saccharide produced in step (f) are isolated and thereafter regenerated in a reaction environment.

In a preferred embodiment, the process according to the invention is characterized in that:
the nucleoside monophosphate is a monophosphorylated conjugate of a ribose or a deoxyribose with uracil; preferably uridine monophosphate; and/or
the nucleoside monophosphate is employed in a concentration of at least 0.125 mM; preferably at least 0.25; preferably at least 0.5 mM; and/or
the phosphate donor is a mono- or polyphosphate of a nucleoside; preferably a nucleoside triphosphate; more preferably adenosine triphosphate;and/or
the phosphate donor is employed in a concentration which is at least as high as the concentration of the nucleoside monophosphate; and/or
in step (b) the phosphate donor is not regenerated.

In other preferred embodiments the process according to the invention is characterized in that
the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase); and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) consisting of an amino acid sequence selected from the group of sequences consisting of SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the first glycosyl transferase is preferably a sucrose synthase; preferably a uridine diphosphate specific sucrose synthase belonging to EC class 2.4.1.13; and/or
the second glycosyltransferase is preferably uridine diphosphate dependent glycosyltransferase.

In other preferred embodiments the process according to the invention is characterized in that
the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase) belonging to EC class 2.7.4.22 or EC 2.7.4.14; and/or
the nucleoside monophosphate kinase comprises a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or
the first glycosyl transferase is preferably a sucrose synthase; preferably a uridine diphosphate specific sucrose synthase belonging to EC class 2.4.1.13; and/or
the second glycosyltransferase is preferably uridine diphosphate dependent glycosyltransferase.

In preferred embodiments of the process according to the invention
all or at least one enzyme is subjected to a purification step prior to being used; and/or
the nucleoside monophosphate kinase, and/or the first glycosyltransferase and/or the second glycosyltransferase, and/or the third glycosyltransferase the epimerase and/or the third glycosyltransferase has been produced in genetically modified organism comprising a genetic modification involving deletion of one or more genes encoding for a polypeptide having nucleotide diphosphate-sugar hydrolase activity.

In a preferred embodiment, the process according to the invention is characterized in that:
the glycosylated organic compound is rebaudioside M (reb M); and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is employed in a concentration of at least 1.5 mU/ml; preferably of at least 4.5 mU/ml; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2; and/or
the nucleoside diphosphate saccharide is uridine diphosphate glucose (UDP-glucose); and/or
the organic compound having a nucleophilic group is rebaudioside A (reb A); and/or
the organic compound having a nucleophilic group is employed a concentration of about 40 mM; and/or
the second glycosyltransferase is an uridine diphosphate dependent glycosyltransferases (UDP-glycosyltransferases) comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4; and/or
the third glycosyltransferase is an uridine diphosphate dependent glycosyltransferases (UDP-glycosyltransferases) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7; and/or
the phosphate donor and the nucleoside monophosphate are employed in a total concentration within the range 0.5 mM to 2.0 mM; and/or
the nucleoside monophosphate and the phosphate donor are employed in a molar ratio within the range 0.6 o 1.5, such as 1.0±0.5, and most preferably the nucleoside monophosphate and the phosphate donor are employed in equimolar amount, i.e. in a molar ratio of 1.0; and/or
the process is carried out at a temperature of around 45° C.; and/or
the process is carried out at a pH of about 6.5; and/or
the process is carried out within a total reaction time of about 41.5 h; and/or
the saccharide donor is added to the reaction in a concentration of about 1000 mM; and/or
in step (b) the phosphate donor is not regenerated.

In a preferred embodiment, the process according to the invention is characterized in that:
the glycosylated organic compound is rebaudioside M (reb M); and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or
the nucleoside monophosphate kinase is employed in a concentration of at least 1.5 mU/ml; preferably of at least 4.5 mU/ml; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2; and/or
the nucleoside diphosphate saccharide is uridine diphosphate glucose (UDP-glucose); and/or
the organic compound having a nucleophilic group is rebaudioside A (reb A); and/or
the organic compound having a nucleophilic group is employed a concentration of about 40 mM; and/or
the second glycosyltransferase is an uridine diphosphate dependent glycosyltransferases (UDP-glycosyltransferases) comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4; and/or
the third glycosyltransferase is an uridine diphosphate dependent glycosyltransferases (UDP-glycosyltransferases) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7; and/or
the phosphate donor and the nucleoside monophosphate are employed in a total concentration within the range 0.5 mM to 2.0 mM; and/or
the nucleoside monophosphate and the phosphate donor are employed in a molar ratio within the range 0.6 o 1.5, such as 1.0±0.5, and most preferably the nucleoside monophosphate and the phosphate donor are employed in equimolar amount, i.e. in a molar ratio of 1.0; and/or
the process is carried out at a temperature of around 45° C.; and/or
the process is carried out at a pH of about 6.5; and/or
the process is carried out within a total reaction time of about 41.5 h; and/or
the saccharide donor is added to the reaction in a concentration of about 1000 mM; and/or
in step (b) the phosphate donor is not regenerated.

In other preferred embodiments the process according to the invention is characterized in that:
the glycosylated organic compound is Glc-polydatin; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the organic compound having a nucleophilic group is polydatin; and/or
the organic compound having a nucleophilic group is employed a concentration of about 10 mM; and/or
the second glycosyltransferase is an uridine diphosphate dependent glycosyltransferase (UDP-glycosyltransferase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5; and/or
the phosphate donor and the nucleoside monophosphate are employed in a total concentration within the range 0.2 mM to 2 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or
the process is carried out at a temperature of around 40° C.; and/or
the process is carried out at a pH of about 6.5; and/or
the process is carried out within a total reaction time of about 71 h; and/or
the saccharide donor is added to the reaction in a concentration of about 750 mM and/or
in step (b) the phosphate donor is not regenerated.

In other preferred embodiments the process according to the invention is characterized in that:
the glycosylated organic compound is Glc-polydatin; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the organic compound having a nucleophilic group is polydatin; and/or
the organic compound having a nucleophilic group is employed a concentration of about 10 mM; and/or
the second glycosyltransferase is an uridine diphosphate dependent glycosyltransferase (UDP-glycosyltransferase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5; and/or
the phosphate donor and the nucleoside monophosphate are employed in a total concentration within the range 0.2 mM to 2 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0;and/or
the process is carried out at a temperature of around 40° C.; and/or
the process is carried out at a pH of about 6.5; and/or
the process is carried out within a total reaction time of about 71 h; and/or
the saccharide donor is added to the reaction in a concentration of about 750 mM and/or
in step (b) the phosphate donor is not regenerated.

In other preferred embodiments, the process according to the invention is characterized in that:
the glycosylated organic compound is Lacto-N-neotetraose; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the glucose moiety of nucleoside diphosphate saccharide is converted into a galactose moiety under catalysis of a glucose galactose epimerase wherein the glucose galactose epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8; and/or
the organic compound having a nucleophilic group is Lacto-N-triose II; and/or
the organic compound having a nucleophilic group is employed a concentration of about 100 mM; and/or
the second glycosyltransferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6; and/or
the phosphate donor and the nucleoside monophosphate are employed in a total concentration within the range 2 mM to 4 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or
the process is carried out at a temperature of around 30° C.; and/or
the process is carried out at a pH of about 7.5; and/or
the process is carried out within a total reaction time of about 72 h; and/or
the saccharide donor is added to the reaction in a concentration of about 500 mM; and/or
in step (b) the phosphate donor is not regenerated.

In other preferred embodiments, the process according to the invention is characterized in that:
the glycosylated organic compound is Lacto-N-neotetraose; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the glucose moiety of nucleoside diphosphate saccharide is converted into a galactose moiety under catalysis of a glucose galactose epimerase wherein the glucose galactose epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8; and/or
the organic compound having a nucleophilic group is Lacto-N-triose II; and/or
the organic compound having a nucleophilic group is employed a concentration of about 100 mM; and/or
the second glycosyltransferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6; and/or
the phosphate donor and the nucleoside monophosphate are employed in a total concentration within the range 2 mM to 4 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or
the process is carried out at a temperature of around 30° C.; and/or
the process is carried out at a pH of about 7.5; and/or
the process is carried out within a total reaction time of about 72 h; and/or
the saccharide donor is added to the reaction in a concentration of about 500 mM; and/or
in step (b) the phosphate donor is not regenerated.

Another aspect of the invention relates to a composition comprising a (i) glycosylated organic compound obtainable by the process according to the invention as described above in combination with (ii) a nucleoside monophosphate kinase, or a first glycosyltransferase, a second glycosyltransferase, or a third glycosyltransferase, an epimerase, or any combination of the foregoing.

Preferably, said glycosylated organic compound is present in an amount within the range of from 2 to 99 mol % calculated with respect to the molar amount of unreacted organic compound having a nucleophilic group or reaction intermediates and side products.

Preferably, the glycosylated organic compound is a steviol glycoside selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside 1l, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3,rebaudioside C4, rebaudioside C5, rebaudioside C6, rebaudioside D2, rebaudioside D3, rebaudioside D4, rebaudioside D5, rebaudioside D6, rebaudioside D8, rebaudioside E5,rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside I2, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3,rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2020112957A1 which is hereby incorporated by reference, synthetic steviol glycosides, and combinations thereof; preferably rebaudioside M.

In another preferred embodiment, the glycosylated organic compound is a glycosylated polyphenol.

In preferred embodiments, the glycosylated organic compound is a polysaccharide.

Another aspect of the invention relates to the use of an enzyme having a glycosyltransferase activity in the process according to the invention as described above.

Another aspect of the invention relates to the use of an enzyme having a kinase activity in the process according to the invention as described above.

Another aspect of the invention relates to the use of an enzyme having a epimerase activity in the process according to the invention as described above.

Another aspect of the invention relates to the use of a nucleoside monophosphate to produce a glycosylated organic compound; preferably to produce a glycosylated organic compound in the process according to the invention as described above.

Preferably, said glycosylated organic compound is selected from the group consisting of steviol glycosides, polyphenols and oligosaccharides.

In preferred embodiments, said steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2,rebaudioside C, rebaudioside C2, rebaudioside C3,rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside H2, rebaudioside H3, rebaudioside H4, rebaudioside H5, rebaudioside H6, rebaudioside I2, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2020112957A1 which is hereby incorporated by reference, synthetic steviol glycosides, and combinations thereof; preferably rebaudioside M.

Preferred embodiments of the invention are summarized as clauses 1 to 168 hereinafter:

[clause 1] A process for the preparation of a glycosylated organic compound by in vitro glycosylation of an organic compound having a nucleophilic group with a saccharide under catalysis of a Leloir glycosyltransferase system comprising at least a first glycosyl transferase and a second glycosyl transferase, the process comprising the steps of: (a) providing a nucleoside monophosphate and a phosphate donor; (b) reacting the nucleoside monophosphate and the phosphate donor provided in step (a) under catalysis of a nucleoside monophosphate kinase thereby obtaining a nucleoside diphosphate; (c) providing a saccharide donor; (d) reacting the nucleoside diphosphate ob-tained in step (b) with the saccharide of the saccharide donor provided in step (c) under catalysis of the first glycosyl transferase thereby obtaining a nucleoside diphosphate saccharide; (e) providing the organic compound having a nucleophilic group; and (f) reacting the nucleoside diphosphate saccharide obtained in step (d) with the organic compound having a nucleophilic group provided in step (e) under catalysis of at least the second glycosyltransferase thereby obtaining the glycosylated organic compound.
[clause 2] The process according to clause 1, wherein the phosphate donor and the nucleoside monophosphate are selected independently of one another and are employed in a total concentration of at least 0.05 mM; preferably at least 0.1 mM; more preferably at least 0.2 mM.
[clause 3] The process according to clauses 1 or 2, wherein, the phosphate donor and the nucleoside monophosphate are employed in a total concentration of at most 8.0 mM; preferably at most 4.0 mM.
[clause 4] The process according to any of the proceeding clauses, wherein, the phosphate donor and the nucleoside monophosphate are employed in a total concentration within the range of 0.55±0.5 mM, or 0.6±0.5 mM, or 0.8±0.5 mM, or 1.0±0.5 mM, or 1.2±0.5 mM, or 1.4±0.5 mM, or 1.6±0.5 mM, or 1.8±0.5 mM, or 2.0±0.5 mM, or 2.2±0.5 mM, or 2.4±0.5 mM, or 2.6±0.5 mM, or 2.8±0.5 mM, or 3.0±0.5 mM, or 3.2±0.5 mM, or 3.4±0.5 mM, or 3.6±0.5 mM, or 3.8±0.5 mM, or 4.0±0.5 mM, or 4.2±0.5 mM, or 4.4±0.5 mM, or 4.6±0.5 mM, or 4.8±0.5 mM, 5.0±0.5 mM, or 5.2±0.5 mM, or 5.4±0.5 mM, or 5.6±0.5 mM, or 5.8±0.5 mM, or 6.0±0.5 mM, 6.2±0.5 mM, or 6.4±0.5 mM, or 6.6±0.5 mM, or 6.8±0.5 mM, or 7.0±0.5 mM, or 7.2±0.5 mM; 7.4±0.5 mM, or 7.5±0.5 mM; preferably within the range of 0.1±0.4 mM, or 0.6±0.4 mM, or 0.8±0.4 mM, or 1.0±0.4 mM, or 1.2±0.4 mM, or 1.4±0.4 mM, or 1.6±0.4 mM, or 1.8±0.4 mM, or 2.0±0.4 mM, or 2.2±0.4 mM, or 2.4±0.4 mM, or 2.6±0.4 mM, or 2.8±0.4 mM, or 3.0±0.4 mM, or 3.2±0.4 mM, or 3.4±0.4 mM, or 3.6±0.4 mM, or 3.8±0.4 mM, or 4.0±0.4 mM, or 4.2±0.4 mM, or 4.4±0.4 mM, or 4.6±0.4 mM, or 4.8±0.4 mM, 5.0±0.4 mM, or 5.2±0.4 mM, or 5.4±0.4 mM, or 5.6±0.4 mM, or 5.8±0.4 mM, or 6.0±0.4 mM, 6.2±0.4 mM, or 6.4±0.4 mM, or 6.6±0.4 mM, or 6.8±0.4 mM, or 7.0±0.4 mM, or 7.2±0.4 mM; 7.4±0.4 mM, or 7.5±0.4 mM; more preferably within the range of 0.1±0.3 mM, or 0.6±0.3 mM, or 0.8±0.3 mM, or 1.0±0.3 mM, or 1.2±0.3 mM, or 1.4±0.3 mM, or 1.6±0.3 mM, or 1.8±0.3 mM, or 2.0±0.3 mM, or 2.2±0.3 mM, or 2.4±0.3 mM, or 2.6±0.3 mM, or 2.8±0.3 mM, or 3.0±0.3 mM, or 3.2±0.3 mM, or 3.4±0.3 mM, or 3.6±0.3 mM, or 3.8±0.3 mM, or 4.0±0.3 mM, or 4.2±0.3 mM, or 4.4±0.3 mM, or 4.6±0.3 mM, or 4.8±0.3 mM, 5.0±0.3 mM, or 5.2±0.3 mM, or 5.4±0.3 mM, or 5.6±0.3 mM, or 5.8±0.3 mM, or 6.0±0.3 mM, 6.2±0.3 mM, or 6.4±0.3 mM, or 6.6±0.3 mM, or 6.8±0.3 mM, or 7.0±0.3 mM, or 7.2±0.3 mM; 7.4±0.3 mM, or 7.5±0.3 mM.
[clause 5] The process according to any of the proceeding clauses wherein, the nucleoside monophosphate and the phosphate donor are employed in a molar ratio of at least 0.05; preferably at least 0.3; more preferably at least 0.65.
[clause 6] The process according to any of the proceeding clauses wherein, the nucleoside monophosphate and the phosphate donor are employed in a molar ratio of at most 8.0.
[clause 7] The process according to any of the proceeding clauses wherein, the nucleoside monophosphate and the phosphate donor are employed in a molar ratio within the range 0.05 to 8.0, or 0.1 to 8.0 or 0.8±0.5 to 8.0, or 1.0 to 8.0, or 1.2 to 8.0, or 1.4 to 8.0, or 1.6 to 8.0, or 1.8 to 8.0, or 2.0 to 8.0, or 2.2 to 8.0, or 2.4 to 8.0, or 2.6 to 8.0, or 2.8 to 8.0, or 3.0 to 8.0, or 3.2 to 8.0, or 3.4 to 8.0, or 3.6 to 8.0, or 3.8 to 8.0, or 4.0 to 8.0, or 4.2 to 8.0, or 4.4 to 8.0, or 4.6 to 8.0, or 4.8, 5.0 to 8.0, or 5.2 to 8.0, or 5.4 to 8.0, or 5.6 to 8.0, or 5.8 to 8.0, or 6.0, 6.2 to 8.0, or 6.4 to 8.0, or 6.6 to 8.0, or 6.8 to 8.0, or 7.0 to 8.0 preferably within the range of 0.6 to 4.5, or 0.8 to 4.5, or 1.0 to 4.5, or 1.2 to 4.5, or 1.4 to 4.5, or 1.6 to 4.5, or 1.8 to 4.5, or 2.0 to 4.5, or 2.2 to 4.5, or 2.4 to 4.5, or 2.6 to 4.5, or 2.8 to 4.5, or 3.0 to 4.5 more preferably within the range or 0.6 to 1.5, or 0.8 to 1.5, or 1.0 to 1.5, or 1.2 to 1.5, or 1.4 to 1.5, or 1.6 to 1.5, or 1.8 to 1.5 and even more preferably within the range 0.6 to 1.5 such as 0.6±0.5, or 0.8±0.5, or 1.0±0.5, or 1.2±0.5, or 1.4±0.5.
[clause 8] The process according to any of the proceeding clauses wherein, the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0.
[clause 9] The process according to any of the proceeding clauses, wherein the process is carried out at a pH of at least 2.5; preferably at least 4.5; more preferably of at least 6.5.
[clause 10] The process according to any of the preceding clauses, wherein the process is carried out at a pH of at most 13.5; preferably at most 9.5; more preferably of at most 7.5.
[clause 11] The process according to any of the preceding clauses, wherein the process is carried out at a pH within the range of 3.75±1.25 mM, or 5.75±1.25 mM, or 6.75±1.25 mM, or 7.75±1.25 mM, or 8.75±1.25 mM, or 9.75±1.25 mM, or 10.75±1.25 mM, or 11.75±1.25 mM, or 12.75±1.25 mM preferably within the range of 3.75±1.15 mM, or 5.75±1.15 mM, or 6.75±1.15 mM, or 7.75±1.15 mM, or 8.75±1.15 mM, or 9.75±1.15 mM, or 10.75±1.15 mM, or 11.75±1.15 mM, or 12.75±1.15 mM; more preferably within the range of 3.75±1.05 mM, or 5.75±1.05 mM, or 6.75±1.05 mM, or 7.75±1.05 mM, or 8.75±1.05 mM, or 9.75±1.05 mM, or 10.75±1.05 mM, or 11.75±1.05 mM, or 12.75±1.05 mM.
[clause 12] The process according to any of the preceding clauses, wherein the process is carried out at a temperature of at least 20° C.; preferably at least 25° C.; more preferably of at least 30° C.
[clause 13] The process according to any of the preceding clauses, wherein the process is carried out at a temperature of at most 100° C., or at most 65° C.; preferably at most 55° C.; more preferably of at most 45° C.
[clause 14] The process according to any of the preceding clauses, wherein the process is carried out at a temperature within the range of 28±8° C., or 36±8° C., or 44±8° C., or 52±8° C., or 60±8° C., or 68±8° C., or 76±8° C., or 84±8° C.; 92±8° C. preferably within the range of 28±7° C., or 36±7° C., or 44±7° C., or 52±8° C., or 60±7° C., or 68±7° C., or 76±7° or 84±7° C.; 92±7° C.; more preferably within the range of 28±6° C., or 36±6° C., or 44±6° C., or 52±6° C., or 60±6° C., or 68±6° C., or 76±6° C., or 84±6° C.; 92±6° C.
[clause 15] The process according to any of the preceding clauses, wherein the process is carried out within a total reaction time comprised within the range of from 0.5 to 120 h; preferably from 24 to 120 h; more preferably from 40 to 120 h.
[clause 16] The process according to any of the preceding clauses, wherein the phosphate donor differs from the nucleoside monophosphate.
[clause 17] The process according to any of the preceding clauses, wherein the phosphate donor and the nucleoside monophosphate donor present different molecular weights.
[clause 18] The process according to any of the preceding clauses, wherein the phosphate donor and the nucleoside monophosphate present differ by at least one carbon atom.
[clause 19] The process according to any of the preceding clauses, wherein the phosphate donor and the nucleoside monophosphate present differ by at most one carbon atom.
[clause 20] The process according to any of the preceding clauses, wherein the phosphate donor differs from the nucleoside monophosphate in the nucleobase and/or in the number of the phosphate groups.
[clause 21] The process according to any of the preceding clauses, wherein the process is performed as a batch process.
[clause 22] The process according to any of the preceding clauses, wherein the process is performed as a continuous or as a semi-continuous process.
[clause 23] The process according to any of the preceding clauses, wherein at least one of steps (a), (b), (c), (d), (e) and (f) is repeated at least once.
[clause 24] The process according to any of the preceding clauses, wherein at least two of steps (a), (b), (c), (d), (e) and (f) are performed in a single reactor.
[clause 25] The process according to any of the preceding clauses, wherein all steps (a), (b), (c), (d), (e) and (f) are performed in a single reactor.
[clause 26] The process according to any of the preceding clauses, wherein steps (a), (c) and/or (e) are performed simultaneously.
[clause 27] The process according to any of the preceding clauses, wherein steps (b), (d) and/or (f) are performed simultaneously.
[clause 28] The process according to any of the preceding clauses, which comprises a further step of (g) purifying the glycosylated organic compound.
[clause 29] The process according to any of the preceding clauses, preferably to clause 28, wherein step (g) involves a microfiltration step, an ion-exchange step, a crystallization step, or any combination of the foregoing.
[clause 30] The process according to any of the preceding clauses, preferably to any of clauses 28 or 29, wherein step (g) involves at least a crystallization step and wherein the crystallization step comprises dissolving the reaction mixture of step (f) in an appropriate first solvent and precipitating at least the glycosylated organic compound.
[clause 31] The process according to any of the preceding clauses, preferably to clause 30, wherein the precipitation step involves lowering the temperature.
[clause 32] The process according to any of the preceding clauses, preferably to any of clauses 30 or 31, wherein the precipitation step involves addition of a second solvent wherein at least one component of the reaction mixture obtained in step (f) presents lower solubility than in said first solvent.
[clause 33] The process according to any of the preceding clauses, preferably to any of clauses 28 to 31, wherein the process involves involving re-crystallization of the glycosylated organic compound by solvent evaporation.
[clause 34] The process according to any of the preceding clauses, wherein the nucleoside monophosphate is not adenosine monophosphate.
[clause 35] The process according to any of the preceding clauses, wherein the nucleoside monophosphate is a monophosphorylated conjugate of a ribose or a deoxyribose with a nucleobase selected from the group consisting of adenine, guanine, inosine, cytosine, thymine, and uracil.
[clause 36] The process according to any of the preceding clauses, wherein the nucleoside monophosphate is a monophosphorylated conjugate of a ribose or a deoxyribose with a pyrimidine base selected from the group consisting of cytosine, thymine, and uracil.
[clause 37] The process according to any of the preceding clauses, wherein the nucleoside monophosphate is a monophosphorylated conjugate of a ribose or a deoxyribose with uracil; preferably uridine monophosphate.
[clause 38] The process according to any of the preceding clauses, wherein, the nucleoside monophosphate is employed in a concentration of at least 0.012 mM; preferably at least 0.05 mM; more preferably at least 0.1 mM.
[clause 39] The process according to any of the preceding clauses, wherein, the nucleoside monophosphate is employed in a concentration of at most 0.5 mM; preferably at most 4 mM; more preferably at most 2.0 mM.
[clause 40] The process according to any of the preceding clauses, wherein, the nucleoside monophosphate is employed in a concentration within the range of 0.382±0.37 mM, or 0.4±0.37 mM, or 0.46±0.37 mM, or 0.5±0.37 mM, or 0.56±0.37 mM, or 0.62±0.37 mM, or 0.68±0.37 mM, or 0.74±0.37 mM, or 0.8±0.37 mM, or 0.88±0.37 mM, or 1.48±0.37 mM, or 1.48±0.37 mM, or 1.63±0.37 mM, or 1.78±0.37 mM, or 1.93±0.37 mM, or 2.08±0.37 mM, or 2.23±0.37 mM, or 2.38±0.37 mM, or 2.53±0.37 mM, or 2.68±0.37 mM, or 2.83±0.37 mM, or 2.98±0.37 mM, or 3.13±0.37 mM, or 3.28±0.37 mM, or 3.43±0.37 mM, or 3.58±0.37 mM, or 3.63±0.37 mM; preferably within the range of 0.382±0.27 mM, or 0.4±0.27 mM, or 0.46±0.27 mM, or 0.5±0.27 mM, or 0.56±0.27 mM, or 0.62±0.27 mM, or 0.68±0.27 mM, or 0.74±0.27 mM, or 0.8±0.27 mM, or 0.88±0.27 mM, or 1.48±0.27 mM, or 1.48±0.27 mM, or 1.63±0.27 mM, or 1.78±0.27 mM, or 1.93±0.27 mM, or 2.08±0.27 mM, or 2.23±0.27 mM, or 2.38±0.27 mM, or 2.53±0.27 mM, or 2.68±0.27 mM, or 2.83±0.27 mM, or 2.98±0.27 mM, or 3.13±0.27 mM, or 3.28±0.27 mM, or 3.43±0.27 mM, or 3.58±0.27 mM, or 3.63±0.27 mM; more preferably within the range of 0.382±0.27 mM, or 0.4±0.17 mM, or 0.46±0.17 mM, or 0.5±0.17 mM, or 0.56±0.17 mM, or 0.62±0.17 mM, or 0.68±0.17 mM, or 0.74±0.17 mM, or 0.8±0.17 mM, or 0.88±0.17 mM, or 1.48±0.17 mM, or 1.48±0.17 mM, or 1.63±0.17 mM, or 1.78±0.17 mM, or 1.93±0.17 mM, or 2.08±0.17 mM, or 2.23±0.17 mM, or 2.38±0.17 mM, or 2.53±0.17 mM, or 2.68±0.17 mM, or 2.83±0.17 mM, or 2.98±0.17 mM, or 3.13±0.17 mM, or 3.28±0.17 mM, or 3.43±0.17 mM, or 3.58±0.17 mM, or 3.63±0.17 mM.
[clause 41] The process according to any of the preceding clauses, wherein the phosphate donor is selected from the group consisting of organic monophosphates, organic polyphosphates, inorganic monophosphates and inorganic polyphosphates.
[clause 42] The process according to any of the preceding clauses, wherein the phosphate donor is selected from the group consisting of nucleoside monophosphates, nucleoside polyphosphates, creatine monophosphate, creatine polyphosphate, and phosphoenolpyruvic acid.
[clause 43] The process according to any of the preceding clauses, wherein the phosphate donor is a mono- or polyphosphate of a nucleoside; preferably a nucleoside triphosphate; more preferably adenosine triphosphate.
[clause 44] The process according to any of the preceding clauses, wherein the phosphate donor is employed in a concentration which is at least as high as the concentration of the nucleoside monophosphate.
[clause 45] The process according to any of the preceding clauses, wherein the phosphate donor is employed in a concentration that is relatively at least 0.1 mM greater than the concentration of the nucleoside monophosphate.
[clause 46] The process according to any of the preceding clauses, wherein the phosphate donor is employed in a concentration of at least 0.012 mM; preferably at least 0.05 mM; more preferably at least 0.1 mM.
[clause 47] The process according to any of the preceding clauses, wherein phosphate donor is employed in a concentration of at most 0.5 mM; preferably at most 4 mM; more preferably at most 2.0 mM.
[clause 48] The process according to any of the preceding clauses, wherein phosphate donor is employed in a concentration within the range of 0.382±0.37 mM, or 0.4±0.37 mM, or 0.46±0.37 mM, or 0.5±0.37 mM, or 0.56±0.37 mM, or 0.62±0.37 mM, or 0.68±0.37 mM, or 0.74±0.37 mM, or 0.8±0.37 mM, or 0.88±0.37 mM, or 1.48±0.37 mM, or 1.48±0.37 mM, or 1.63±0.37 mM, or 1.78±0.37 mM, or 1.93±0.37 mM, or 2.08±0.37 mM, or 2.23±0.37 mM, or 2.38±0.37 mM, or 2.53±0.37 mM, or 2.68±0.37 mM, or 2.83±0.37 mM, or 2.98±0.37 mM, or 3.13±0.37 mM, or 3.28±0.37 mM, or 3.43±0.37 mM, or 3.58±0.37 mM, or 3.63±0.37 mM; preferably within the range of 0.382±0.27 mM, or 0.4±0.27 mM, or 0.46±0.27 mM, or 0.5±0.27 mM, or 0.56±0.27 mM, or 0.62±0.27 mM, or 0.68±0.27 mM, or 0.74±0.27 mM, or 0.8±0.27 mM, or 0.88±0.27 mM, or 1.48±0.27 mM, or 1.48±0.27 mM, or 1.63±0.27 mM, or 1.78±0.27 mM, or 1.93±0.27 mM, or 2.08±0.27 mM, or 2.23±0.27 mM, or 2.38±0.27 mM, or 2.53±0.27 mM, or 2.68±0.27 mM, or 2.83±0.27 mM, or 2.98±0.27 mM, or 3.13±0.27 mM, or 3.28±0.27 mM, or 3.43±0.27 mM, or 3.58±0.27 mM, or 3.63±0.27 mM; more preferably within the range of 0.382±0.27 mM, or 0.4±0.17 mM, or 0.46±0.17 mM, or 0.5±0.17 mM, or 0.56±0.17 mM, or 0.62±0.17 mM, or 0.68±0.17 mM, or 0.74±0.17 mM, or 0.8±0.17 mM, or 0.88±0.17 mM, or 1.48±0.17 mM, or 1.48±0.17 mM, or 1.63±0.17 mM, or 1.78±0.17 mM, or 1.93±0.17 mM, or 2.08±0.17 mM, or 2.23±0.17 mM, or 2.38±0.17 mM, or 2.53±0.17 mM, or 2.68±0.17 mM, or 2.83±0.17 mM, or 2.98±0.17 mM, or 3.13±0.17 mM, or 3.28±0.17 mM, or 3.43±0.17 mM, or 3.58±0.17 mM, or 3.63±0.17 mM;
[clause 49] The process according to any of the preceding clauses, wherein the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase)
[clause 50] The process according to any of the preceding clauses, wherein the nucleoside monophosphate kinase is an uridylate kinase (UMP-kinase) belonging to EC class 2.7.4.22.
[clause 51] The process according to any of clauses 1 to 49, wherein the nucleoside monophosphate kinase is an uridylate kinase (UMP-kinase) belonging to EC class EC 2.7.4.14.
[clause 52] The process according to any of clauses 1 to 49, wherein the nucleoside monophosphate kinase is an uridylate kinase comprising or consisting of an amino acid sequence selected from the group of sequences consisting of SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.
[clause 53] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1.
[clause 54] The process according to any of the preceding clauses, preferably to any of clauses 1 to 49, wherein the nucleoside monophosphate kinase is an uridylate kinase (UMP-kinase) comprising or consisting of an amino acid sequence corresponding to SEQ ID NO: 1.
[clause 55] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 10.
[clause 56] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 11.
[clause 57] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 12.
[clause 58] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 13.
[clause 59] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 14.
[clause 60] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 15.
[clause 61] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 16.
[clause 62] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 17.
[clause 63] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 18.
[clause 64] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 19.
[clause 65] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 20.
[clause 66] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 21.
[clause 67] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 22.
[clause 68] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 23.
[clause 69] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 24.
[clause 70] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 25.
[clause 71] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 26.
[clause 72] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 27.
[clause 73] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 28.
[clause 74] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 29.
[clause 75] The process according to any of the preceding clauses, preferably to any of clauses 1 to 49, wherein the nucleoside monophosphate kinase is an uridylate kinase (UMP-kinase) comprising a primary sequence having a sequence identity of at least 60%, or at least 65%, or at least 70%, or at least 75%, or at least 80%, or at least 85%, or at least 90%, or at least 93%, or at least 94%, at least 95%, or at least 96%, or at least 97%; and in particular of at least 98%, or at least 99%, or 100%; preferably of at least 85%, or at least 87%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 30.
[clause 76] The process according to any of the preceding clauses, wherein the nucleoside monophosphate kinase is employed in a concentration of at least 0.05 mU/ml; preferably of at least 0.1 mU/ml; preferably of at least 0.2 mU/ml; preferably of at least 0.3 mU/ml; preferably of at least 0.4 mU/ml; preferably of at least 0.5 mU/ml; preferably of at least 0.6; preferably of at least 0.7 mU/ml; preferably of at least 0.8 mU/ml; preferably of at least 0.9 mU/ml; more preferably of at least 1.0 mU/ml.
[clause 77] The process according to any of the preceding clauses, wherein the saccharide donor is a, disaccharide, oligosaccharide, or polysaccharide.
[clause 78] The process according to any of the preceding clauses, wherein the saccharide donor is or comprises a moiety derived from galactose, glucose, fucose, mannose, glucuronic acid, sialyic acid, N-acetylgalactosamine, N-acetylglucosamin, tagatose, talose, xylose, arabinose, rhamnose, starch, or inulin.
[clause 79] The process according to any of the preceding clauses, wherein the saccharide donor is or comprises a moiety derived from galactose and/or glucose; preferably sucrose.
[clause 80] The process according to any of the preceding clauses, wherein the saccharide donor is added to the reaction in a concentration range of from 100 mM up to 2000 mM, 200 mM up to 2000 mM, 300 mM up to 2000 mM, 400 mM up to 2000 mM, 500 mM up to 2000 mM, 600 mM up to 2000 mM, 700 mM up to 2000 mM, 800 mM up to 2000 mM, 900 mM up to 2000 mM, 1000 mM up to 2000 mM, 1100 mM up to 2000 mM, 1200 mM up to 2000 mM, 1300 mM up to 2000 mM, 1400 mM up to 2000 mM, 1500 mM up to 2000 mM, 1600 mM up to 2000 mM, 1700 mM up to 2000 mM, 1800 mM up to 2000 mM, 1900 mM up to 2000 mM; preferably in the range of 500 mM up to 1100 mM, 600 mM up to 1100 mM, 700 mM up to 1100 mM.
[clause 81] The process according to any of the preceding clauses, wherein the first glycosyl transferase is a sucrose synthase; preferably a uridine diphosphate specific sucrose synthase
[clause 82] The process according to any of the preceding clauses, wherein the first glycosyl transferase is a sucrose synthase belonging to EC class 2.4.1.13.
[clause 83] The process according to any of the preceding clauses, wherein the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a wild-type polypeptide sequence of Arabidopsis thaliana preferably a wild-type polylpeptide sequence of Arabidopsis thaliana corresponding to SEQ ID NO: 9.
[clause 84] The process according to any of the preceding clauses, wherein the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2.
[clause 85] The process according to any of the preceding clauses, wherein the first glycosyl transferase is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3.
[clause 86] The process according to any of the preceding clauses, wherein first glycosyl transferase is employed is employed in a concentration of at least 5 mU/ml; preferably of at least 10 mU/ml; preferably of at least 20 mU/ml; preferably of at least 25 mU/ml; preferably of at least 30 mU/ml; preferably of at least 35 mU/ml; preferably of at least 40 mU/ml; preferably of at least 45 mU/ml; more preferably of at least 50 mU/ml.
[clause 87] The process according to any of the preceding clauses, wherein the nucleophilic group of the organic compound having a nucleophilic group is selected from the group consisting of —OH, —NH₂, —PH and —SH.
[clause 88] The process according to any of the preceding clauses, wherein the organic compound having a nucleophilic group is selected from the group consisting of terpenes, steroids, carotenoids, peptides, proteins, antibodies, sweeteners, steviol glycosides, rebaudiosides, polyphenols, oligosaccharides, and polysaccharides.
[clause 89] The process according to any of the preceding clauses, preferably to any of clauses 1 to 88 wherein the organic compound having a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5,rebaudioside E7, rebaudioside F, rebaudioside F2, rebaudioside F3, rebaudioside H, rebaudioside 112, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4,rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2018213279A1 which is hereby incorporated by reference, synthetic steviol glycosides, and combinations thereof; preferably rebaudioside A.
[clause 90] The process according to any of the preceding clauses, preferably to any of clauses 1 to 88, wherein the organic compound having a nucleophilic group is a polyphenol; preferably a mono or polysubstituted stilbenoide glucoside; more preferably polydatin.
[clause 91] The process according to any of the preceding clauses, preferably to any of clauses 1 to 88, wherein the organic compound having a nucleophilic group is an oligosaccharide; preferably an oligosaccharide containing three saccharide units; more preferably, Lacto-N-triose II.
[clause 92] The process according to any of the preceding clauses, wherein the organic compound having a nucleophilic group is employed a concentration of at least 1 mM; preferably at least 1.5 mM; preferably at least 2.0 mM.
[clause 93] The process according to any of the preceding clauses, wherein the organic compound having a nucleophilic group is employed a concentration of at most 500 mM; preferably at most 100 mM more preferably at most 10 mM.
[clause 94] The process according to any of the preceding clauses, wherein the organic compound having a nucleophilic group is employed a concentration within the range of from 1 to 500 mM, or 2 mM to 500 mM, or 3 mM to 500 mM, or 4 mM to 500 mM, or 5 mM to 500 mM, or 6 mM to 500 mM, or 7 mM to 500 mM, or 8 mM to 500 mM, or 9 mM to 500 mM; preferably from 10 mM to 500 mM; more preferably from 40 mM to 500 mM, or 50 mM to 500 mM, or 60 mM to 100 mM, or 70 mM to 500 mM, or 80 mM to 500 mM, or 90 mM to 500; and more preferably from 100 to 500 mM.
[clause 95] The process according to any of the preceding clauses, wherein the second glycosyltransferase is a nucleotide sugar-dependent glycosyltransferase.
[clause 96] The process according to any of the preceding clauses, wherein the second glycosyltransferase is selected from the group consisting of uridine diphosphate glycosyltransferases, adenosine diphosphate glycosyltransferases, cytidine diphosphate glycosyltransferase, guanosine diphosphate glycosyltransferase, thymidine diphosphate glycosyltransferase; preferably an uridine diphosphate dependent glycosyltransferase.
[clause 97] The process according to any of the preceding clauses, wherein the second glycosyltransferase is capable of catalyzing the transfer of a sugar moiety from a uridine diphosphate sugar onto the nucleophilic group of the organic compound.
[clause 98] The process according to any of the preceding clauses, wherein the second glycosyltransferase is capable of catalyzing the transfer of a sugar moiety onto one or more position of an organic compound having a nucleophilic group wherein the organic compound presenting a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2,rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside 112, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4,rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3,rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2018213279A1 which is hereby incorporated by reference, synthetic steviol glycosides, and combinations thereof.
[clause 99] The process according to any of the preceding clauses, wherein the second glycosyl transferase is a uridine diphosphate dependent glycosyltransferase belonging to EC class 2.4.1X.
[clause 100] The process according to any of the preceding clauses, preferably to any of clauses 1 to 98, wherein the second glycosyl transferase is a uridine diphosphate dependent glycosyltransferase comprising a primary sequence selected from the listing of GenInfo identifier numbers presented in List 1 as described above.
[clause 101] The process according to any of clause 1 to 98 wherein the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to any of the polypeptide sequence of List 1 as described above.
[clause 102] The process according to any of the preceding clauses, preferably to any of clauses 1 to 98, wherein the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to a wild type polypeptide sequence of Solanum lycopersicum; preferably a wild-type polylpeptide sequence of Solanum lycopersicum having NCBI accession number XP_004250485.1.
[clause 103] The process according to any of the preceding clauses, preferably to any of clauses 1 to 98 or clauses 74 to 75, wherein the second glycosyl transferase is a uridine diphosphate dependent glycosyltransferase comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4.
[clause 104] The process according to any of the preceding clauses, wherein the second glycosyltransferase is capable of catalyzing glycosylation of a polyphenol; preferably a mono di- or polysubstituted stilbenoide glucoside; more preferably polydatin.
[clause 105] The process according to any of the preceding clauses, preferably to any of clauses 1 to 98 or clauses 77, wherein the second glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5.
[clause 106] The process according to any of the preceding clauses, wherein the second glycosyltransferase is capable of catalyzing glycosylation of oligosaccharide preferably an oligosaccharide containing three saccharide units; more preferably, Lacto-N-triose II.
[clause 107] The process according to any of the preceding clauses, preferably to any of clauses 1 to 70 or clause79, wherein the second glycosyl transferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6.
[clause 108] The process according to any of the preceding clauses, wherein the second glycosyltransferase is employed in a concentration of at least 0.2 mU/ml; preferably of at least 0.4 mU/ml; preferably of at least 0.6 mU/ml; preferably of at least 0.8 mU/ml; preferably of at least 1.0 mU/ml; preferably of at least 1.2 mU/ml; preferably of at least 1.4 mU/ml; preferably of at least 1.6 mU/ml; preferably of at least 1.8 mU/ml; preferably of at least 2.0 mU/ml; preferably of at least 2.2 mU/ml; preferably of at least 2.4 mU/ml; preferably of at least 2.6 mU/ml; preferably of at least 2.8 mU/ml; preferably of at least 3.0 mU/ml; preferably of at least 3.2 mU/ml; preferably of at least 3.3 mU/ml.
[clause 109] The process according to any of the preceding clauses, wherein the process involves the use in step (f) of a third glycosyltransferase and wherein said third glycosyltransferase is a nucleotide sugar-dependent glycosyltransferase.
[clause 110] The process according to any of the preceding clauses, preferably to clause 109, wherein the third glycosyltransferase is selected from the group consisting of uridine diphosphate glycosyltransferases, adenosine diphosphate glycosyl-transferases, cytidine diphosphate glycosyltransferase, guanosine diphosphate glycosyltransferase, thymidine diphosphate glycosyltransferase; preferably an uridine diphosphate dependent glycosyltransferase.
[clause 111] The process according to any of the preceding clauses, preferably to any of clauses 109 and 110, wherein the third glycosyltransferase is capable of catalyzing the transfer of a sugar moiety onto one or more position of an organic compound having a nucleophilic group wherein the organic compound presenting a nucleophilic group is selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside 112, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4,rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2018213279A1 which is hereby incorporated by reference.
[clause 112] The process according to any of the preceding clauses, preferably to any of clauses 109 to 111, wherein the third glycosyl transferase is a uridine diphosphate dependent glycosyltransferase belonging to EC class 2.4.1X.
[clause 113] The process according to any of the preceding clauses, preferably to any of clauses 82 to 84, wherein the third glycosyl transferase is a uridine diphosphate dependent glycosyltransferase comprising a primary sequence selected from the listing of GenInfo identifier numbers presented in List 1 as described above.
[clause 114] The process according to any of the preceding clauses, preferably to any of clauses 109 to 111, wherein the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 97%; and in particular at least 98%, or at least 99%, or 100%, in each case to any of the polypeptide sequence of List 1 as described above.
[clause 115] The process according to any of the preceding clauses, preferably to any of the clauses 109 to 111, wherein the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to a wild type polypeptide sequence of Stevia rebaudiana preferably a wild-type polylpeptide sequence of Stevia rebaudiana having GenBank number AAR06912.1.
[clause 116] The process according to any of clauses 109 to 111 or 113, wherein the third glycosyl transferase is a uridine diphosphate dependent glycosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7.
[clause 117] The process according to any of the preceding clauses, preferably to any of clauses 82 to 89, wherein the third glycosyltransferase is employed in a concentration of at least 1.0 mU/ml; preferably of at least 2.0 mU/ml; preferably of at least 3.0 mU/ml; preferably of at least 4.0 mU/ml; preferably of at least 5.0 mU/ml; preferably of at least 6.0 mU/ml; preferably of at least 7.0 mU/ml; preferably of at least 8.0 mU/ml; preferably of at least 9.0 mU/ml; preferably of at least 10 mU/ml; preferably of at least 11 mU/ml; preferably of at least 12 mU/ml; preferably of at least 13 mU/ml; preferably of at least 14 mU/ml; preferably of at least 15 mU/ml; preferably of at least 16 mU/ml.
[clause 118] The process according to any of the preceding clauses, wherein the nucleoside monophosphate kinase, the first glycosyltransferase, and the second glycosyltransferase are the only enzymes that are employed in the process.
[clause 119] The process according to any of the preceding clauses, wherein the nucleoside diphosphate saccharide obtained in step (d) comprises a sugar moiety and wherein the process comprises the further step of (h) converting the sugar moiety into an epimer thereof under catalysis of an epimerase.
[clause 120] The process according to any of the preceding clauses, preferably to clause 119, wherein the sugar moiety is a glucose moiety that is converted into a galactose moiety, and wherein the epimerase is a glucose galactose epimerase; preferably a UDP-glucose 4-epimerase.
[clause 121] The process according to any of the preceding clauses, preferably to any of clauses 119 and 120, wherein the epimerase is UDP-glucose 4-epimerase belonging to EC class EC 5.1.3.2.
[clause 122] The process according to any of the preceding clauses, preferably to any of clauses 119 and 120, wherein the epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8.
[clause 123] The process according to any of the preceding clauses, wherein at least one enzyme is used as row cell extracts.
[clause 124] The process according to any of the preceding clauses, wherein all or at least one enzyme is subjected to a purification step prior to being used.
[clause 125] The process according to any of the preceding clauses, preferably to clause 124, wherein the at least one enzyme is the nucleoside monophosphate kinase
[clause 126] The process according to any of the preceding clauses, preferably to clause 124, wherein the at least one enzyme is the first glycosyltransferase.
[clause 127] The process according to any of the preceding clauses, preferably to clause 124, wherein the at least one enzyme is the second glycosyltransferase.
[clause 128] The process according to any of the preceding clauses, preferably to clause 124, wherein the at least one enzyme is the epimerase.
[clause 129] The process according to any of the preceding clauses, preferably to clause 124, wherein the at least one enzyme is the third glycosyltransferase.
[clause 130] The process according to any of the preceding clauses, wherein at least one enzyme has been produced in genetically modified organism.
[clause 131] The process according to any of the preceding clauses, preferably of clause 130 wherein, the at least one enzyme that has been produced in genetically modified organism is the first glycosyltransferase according to the present disclosure as defined above.
[clause 132] The process according to any of the preceding clauses, preferably of any of clauses 130 or 131 wherein, said enzyme that has been produced in genetically modified organism is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a wild-type polypeptide sequence of Arabidopsis thaliana; preferably a wild-type polypeptide sequence of Arabidopsis thaliana corresponding to SEQ ID NO: 9.
[clause 133] The process according to any of the preceding clauses, preferably of any of clauses 130 to 132 wherein, said enzyme that has been produced in genetically modified organism is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2.
[clause 134] The process according to any of the preceding clauses, preferably of any of clauses 130 to 133 wherein, said enzyme that has been produced in genetically modified organism is a sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3.
[clause 135] The process according to any of the preceding clauses, preferably of any of clauses 130 to 134, wherein the genetically modified organism is an organism comprising a genetic modification relating to the insertion or deletion of one or more genes in an organism, said organism being selected from the group comprising but not limited to the organism listing presented in List 2 as described above.
[clause 136] The process according to any of the preceding clauses, preferably of any of clauses 130 to 135, wherein the genetically modified organism comprises a genetic modification involving deletion of one or more genes encoding for a polypeptide having nucleotide diphosphate-sugar hydrolase activity.
[clause 137] The process according to any of the preceding clauses, preferably of any of clauses 130 to 136, wherein the genetic modification is a genetic modification resulting in attenuation or elimination of the expression of one or more genes encoding for an enzyme having hydrolase activity by modification of promotor strength, by modification of activating or inhibitory sequences, by modification of ribosome binding sites, by introduction of frame shift mutations, by introduction of premature stop codons or preferably by complete or partial deletion the genes.
[clause 138] The process according to any of the preceding clauses, preferably to clause 137, wherein said hydrolase activity is a 5′-nucleotidase or UDP-sugar hydrolase activity.
[clause 139] The process according to any of the preceding clauses, preferably to any of clauses 137 or 138, wherein said enzyme having hydrolase activity belongs to EC 3.1.3.5 or EC 3.6.1.45 respectively.
[clause 140] The process according to any of the preceding clauses, preferably to any of clauses 137 or 138, wherein said enzyme having hydrolase activity comprises a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a ushA gene product of Escherichia coli parental strain K-12 W3110 having GenBank number BAE76259.1.
[clause 141] The process according to any of the preceding clauses, preferably to any of clauses 130 to 140, wherein the genetically modified organism is Escherichia coli; preferably a genetically modified laboratory derivative of E. coli parental strain K-12 W3110.
[clause 142] The process according to any of the preceding clauses, preferably to clause 141, wherein said E. coli parental strain K-12 W3110 or its laboratory derivative comprises a genetic modification involving deletion of one or more genes encoding for an enzyme having hydrolase activity; preferably a 5′-nucleotidase or UDP-sugar hydrolase activity.
[clause 143] The process according to any of the preceding clauses, preferably to clauses 140 or 141, wherein said E. coli parental strain K-12 W3110 or its laboratory derivative comprises a genetic modification involving deletion of one or more genes encoding for an enzyme having hydrolase activity, said enzyme having hydrolase activity belonging to EC 3.1.3.5 or EC 3.6.1.45 respectively.
[clause 144] The process according to any of the preceding clauses, preferably to any of clauses 140 or 141, wherein said E. coli parental strain K-12 W3110 or its laboratory derivative comprises a genetic modification involving deletion of one or more genes encoding for an enzyme having hydrolase activity, said enzyme having hydrolase activity comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to a protein sequence coded by ushA from Escherichia coli parental strain K-12 W3110 having GenBank number BAE76259.1.
[clause 145] The process according to any of the preceding clauses, preferably to any of clauses 140 to 143, wherein said genetically E. coli parental strain K-12 W3110 or its laboratory derivative comprises a genetic modification resulting in attenuation or elimination of the expression of ushA by modification of promotor strength, by modification of activating or inhibitory sequences, by modification of the ribosome binding site, by introduction of frame shift mutations, by introduction of premature stop codons or preferably by complete or partial deletion of the gene ushA.
[clause 146] The process according to any of the preceding clauses, wherein in step (b) the phosphate donor is not regenerated.
[clause 147] The process according to any of the preceding clauses, preferably to any of clauses 1 to 145, wherein in step (b) a reaction product of the phosphate donor is obtained as a byproduct in step (b) besides the nucleoside diphosphate, whereby the process comprises the further steps of (i) regenerating the reaction product of the phosphate donor thereby obtaining regenerated phosphate donor; and (j) optionally, recirculating at least a portion of the regenerated phosphate donor to step (b).
[clause 148] The process according to any of the preceding clauses, wherein in step (f) a reaction product of the nucleoside diphosphate saccharide is obtained as a byproduct besides the glycosylated organic compound, and wherein the process comprises the further steps of (k) regenerating the reaction product of the nucleoside diphosphate saccharide thereby obtaining regenerated nucleoside monophosphate, regenerated nucleoside diphosphate, or regenerated nucleoside diphosphate saccharide; and (l) optionally, recirculating at least a portion of the regenerated nucleoside monophosphate to step (b), or at least a portion of the regenerated nucleoside diphosphate to step (d), or at least a portion of the regenerated nucleoside diphosphate saccharide to step (f), respectively.
[clause 149] The process according to any of the preceding clauses, wherein the nucleoside monophosphate is a monophosphorylated conjugate of a ribose or a deoxyribose with uracil; preferably uridine monophosphate; and/or wherein the nucleoside monophosphate is employed in a concentration of at least 0.125 mM; preferably at least 0.25; preferably at least 0.5 mM; and/or wherein the phosphate donor is an mono- or polyphosphate of a nucleoside; preferably a nucleoside triphosphate; more preferably adenosine triphosphate; and/or wherein the phosphate donor is employed in a concentration which is at least as high as the concentration of the nucleoside monophosphate; and/or wherein the nucleoside monophosphate is employed in a concentration of at least 0.125 mM; preferably at least 0.25; preferably at least 0.5 mM; and/or wherein in step (b) the phosphate donor is not regenerated.
[clause 150] The process according to any of the preceding clauses, wherein—the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase); and/or—the nucleoside monophosphate kinase is an uridylate kinase (UMP-kinase) belonging to EC class EC 2.7.4.1; and/or—the nucleoside monophosphate kinase is an uridylate kinase (UMP-kinase) belonging to EC class 2.7.4.22; and/or—the nucleoside monophosphate kinase is an uridylate kinase (UMP-kinase) consisting of an amino acid sequence selected from the group of sequences consisting of SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or—the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the first glycosyl transferase is preferably a sucrose synthase; preferably a uridine diphosphate specific sucrose synthase belonging to EC class 2.4.1.13; and/or—the second glycosyltransferase is preferably uridine diphosphate dependent glycosyltransferase.
[clause 151] The process according to any of the preceding clauses, wherein—the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase) belonging to EC class 2.7.4.22 or EC 2.7.4.14; and/or—the nucleoside monophosphate kinase comprises a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or—the first glycosyl transferase is preferably a sucrose synthase; preferably a uridine diphosphate specific sucrose synthase belonging to EC class 2.4.1.13; and/or—the second glycosyltransferase is preferably uridine diphosphate dependent glycosyltransferase.
[clause 152] The process according to any of the preceding clauses, preferably to any of clauses 148 to 151 wherein all or at least one enzyme is subjected to a purification step prior to being used; and/or wherein the nucleoside monophosphate kinase, and/or the first glycosyltransferase and/or the second glycosyltransferase, and/or the third glycosyltransferase and/or the epimerase has been produced in genetically modified organism comprising a genetic modification involving deletion of one or more genes encoding for a polypeptide having nucleotide diphosphate-sugar hydrolase activity.
[clause 153] The process according to any of the preceding clauses, wherein—the glycosylated organic compound is rebaudioside M (reb M); and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or—the nucleoside monophosphate kinase is an uridylate kinase (UMP-kinase) belonging to EC class EC 2.7.4.1; and/or—the nucleoside monophosphate kinase is an uridylate kinase (UMP-kinase) belonging to EC class 2.7.4.22; and/or—the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the nucleoside monophosphate kinase is an uridylate kinase (UMP-kinase) belonging to EC class EC 2.7.4.1; and/or—the nucleoside monophosphate kinase is an uridylate kinase (UMP-kinase) belonging to EC class 2.7.4.22; and/or—the nucleoside monophosphate kinase is employed in a concentration of at least 1.5 mU/ml; preferably of at least 4.5 mU/ml; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2; and/or—the nucleoside diphosphate saccharide is uridine diphosphate glucose (UDP-glucose); and/or—the organic compound having a nucleophilic group is rebaudioside A (reb A); and/or—the organic compound having a nucleophilic group is employed a concentration of about 40 mM; and/or—the second glycosyltransferase is an uridine diphosphate dependent glycosyltransferases (UDP-glycosyltransferases) comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4; and/or—the third glycosyltransferase is an uridine diphosphate dependent glycosyltransferases (UDP-glycosyltransferases) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7; and/or—the phosphate donor and the nucleoside monophosphate are employed in a total concentration within the range 0.5 mM to 2.0 mM; and/or—the nucleoside monophosphate and the phosphate donor are employed in a molar ratio within the range 0.6 o 1.5, such as 1.0±0.5, and most preferably the nucleoside monophosphate and the phosphate donor are employed in equimolar amount, i.e. in a molar ratio of 1.0; and/or—the process is carried out at a temperature of around 45° C.; and/or—the process is carried out at a pH of about 6.5; and/or—the process is carried out within a total reaction time of about 41.5 h; and/or—the saccharide donor is added to the reaction in a concentration of about 1000 mM; and/or—in step (b) the phosphate donor is not regenerated.
[clause 154] The process according to any of the preceding clauses, wherein the glycosylated organic compound is rebaudioside M (reb M); and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or—the nucleoside monophosphate kinase is employed in a concentration of at least 1.5 mU/ml; preferably of at least mU 4.5/ml; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2; and/or—the nucleoside diphosphate saccharide is uridine diphosphate glucose (UDP-glucose); and/or—the organic compound having a nucleophilic group is rebaudioside A (reb A); and/or—the organic compound having a nucleophilic group is employed a concentration of about 40 mM; and/or—the second glycosyltransferase is an uridine diphosphate dependent glycosyltransferases (UDP-glycosyltransferases) comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4; and/or—the third glycosyltransferase is an uridine diphosphate dependent glycosyltransferases (UDP-glycosyltransferases) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7; and/or—the phosphate donor and the nucleoside monophosphate are employed in a total concentration within the range 0.5 mM to 2.0 mM; and/or—the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or—the process is carried out at a temperature of around 45° C.; and/or—the process is carried out at a pH of about 6.5; and/or—the process is carried out within a total reaction time of about 41.5 h; and/or—the saccharide donor is added to the reaction in a concentration of about 1000 mM; and/or—in step (b) the phosphate donor is not regenerated.
[clause 155] The process according to any of the preceding clauses, wherein—the glycosylated organic compound is Glc-polydatin; and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or—the nucleoside monophosphate kinase is an uridylate kinase (UMP-kinase) belonging to EC class EC 2.7.4.1; and/or—the nucleoside monophosphate kinase is an uridylate kinase (UMP-kinase) belonging to EC class 2.7.4.22; and/or—the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or—the nucleoside diphosphate saccharide is uridine diphosphate glucose (UDP-glucose); and/or—the organic compound having a nucleophilic group is polydatin; and/or—the organic compound having a nucleophilic group is employed a concentration of about 10 mM; and/or—the second glycosyltransferase is an uridine diphosphate dependent glycosyltransferase (UDP-glycosyltransferase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5; and/or—the phosphate donor and the nucleoside monophosphate are employed in a total concentration within the range 0.2 mM to 2 mM; and/or—the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0;and/or—the process is carried out at a temperature of around 40° C.; and/or—the process is carried out at a pH of about 6.5; and/or—the process is carried out within a total reaction time of about 71 h; and/or—the saccharide donor is added to the reaction in a concentration of about 750 mM and/or—in step (b) the phosphate donor is not regenerated.
[clause 156] The process according to any of the preceding clauses, wherein—the glycosylated organic compound is Glc-polydatin; and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or—the nucleoside diphosphate saccharide is uridine diphosphate glucose (UDP-glucose); and/or—the organic compound having a nucleophilic group is polydatin; and/or—the organic compound having a nucleophilic group is employed a concentration of about 10 mM; and/or—the second glycosyltransferase is an uridine diphosphate dependent glycosyltransferase (UDP-glycosyltransferase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5; and/or—the phosphate donor and the nucleoside monophosphate are employed in a total concentration within the range 0.2 mM to 2 mM; and/or—the nucleoside monophosphate and the phosphate donor are employed in a molar ratio within the range 0.6to 1.5, such as 1.0±0.5, and most preferably the nucleoside monophosphate and the phosphate donor are employed in equimolar amount, i.e. in a molar ratio of 1.0; and/or—the process is carried out at a temperature of around 40° C.; and/or—the process is carried out at a pH of about 6.5; and/or—the process is carried out within a total reaction time of about 71 h; and/or—the saccharide donor is added to the reaction in a concentration of about 750 mM; and/or—in step (b) the phosphate donor is not regenerated.
[clause 157] The process according to any of the preceding clauses, wherein—the glycosylated organic compound is Lacto-N-neotetraose; and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or—the nucleoside monophosphate kinase is an uridylate kinase (UMP-kinase) belonging to EC class EC 2.7.4.1; and/or—the nucleoside monophosphate kinase is an uridylate kinase (UMP-kinase) belonging to EC class 2.7.4.22; and/or—the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or—the nucleoside diphosphate saccharide is uridine diphosphate glucose (UDP-glucose); and/or—the glucose moiety of nucleoside diphosphate saccharide is converted into a galactose moiety under catalysis of a glucose galactose epimerase wherein the glucose galactose epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8; and/or—the organic compound having a nucleophilic group is Lacto-N-triose II; and/or—the organic compound having a nucleophilic group is employed a concentration of about 100 mM; and/or—the second glycosyltransferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6; and/or—the phosphate donor and the nucleoside monophosphate are employed in a total concentration within the range 2 mM to 4 mM; and/or—the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or—the process is carried out at a temperature of around 30° C.; and/or—the process is carried out at a pH of about 7.5; and/or—the process is carried out within a total reaction time of about 72 h; and/or—the saccharide donor is added to the reaction in a concentration of about 500 mM; and/or—in step (b) the phosphate donor is not regenerated.
[clause 158] The process according to any of the preceding clauses, wherein—the glycosylated organic compound is Lacto-N-neotetraose; and/or—the nucleoside monophosphate is uridine monophosphate (UMP) and the nucleoside diphosphate is uridine diphosphate (UDP); and/or—the phosphate donor is adenosine triphosphate (ATP); and/or—the nucleoside monophosphate kinase is an uridylate kinase (UMP kinase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 1; and/or—the saccharide donor is sucrose; and/or—the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or—the nucleoside diphosphate saccharide is uridine diphosphate glucose (UDP-glucose); and/or—the glucose moiety of nucleoside diphosphate saccharide is converted into a galactose moiety under catalysis of a glucose galactose epimerase wherein the glucose galactose epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8; and/or—the organic compound having a nucleophilic group is Lacto-N-triose II; and/or—the organic compound having a nucleophilic group is employed a concentration of about 100 mM; and/or—the second glycosyltransferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6; and/or—the phosphate donor and the nucleoside monophosphate are employed in a total concentration within the range 2 mM to 4 mM; and/or—the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or—the process is carried out at a temperature of around 30° C.; and/or—the process is carried out at a pH of about 7.5; and/or—the process is carried out within a total reaction time of about 72 h; and/or—the saccharide donor is added to the reaction in a concentration of about 500 mM; and/or in step (b) the phosphate donor is not regenerated.
[clause 159] A composition comprising a (i) glycosylated organic compound obtainable by the process according to any of clauses 1 to 158 in combination with (ii) a nucleoside monophosphate kinase, or a first glycosyltransferase, a second glycosyltransferase, or a third glycosyltransferase an epimerase, or any combination of the foregoing.
[clause 160] The composition according to clause 159, wherein the glycosylated organic compound is a steviol glycoside selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2,rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside 112, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4,rebaudioside Q, rebaudioside Q2, rebaudioside Q3, rebaudioside R, rebaudioside S, rebaudioside T, rebaudioside T1, rebaudioside U, rebaudioside U2, rebaudioside V,rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2018213279A1 which is hereby incorporated by reference, synthetic steviol glycosides, and combinations thereof; preferably rebaudioside M.
[clause 161] The composition according to any of clauses 159 to 160, preferably to clause 159, wherein the glycosylated organic compound is a glycosylated polyphenol.
[clause 162] The composition according to any of clauses 159 to 161, preferably to clause 159, wherein the glycosylated organic compound is a polysaccharide.
[clause 163] Use of an enzyme having a glycosyltransferase activity in the process as defined in any of the previous clauses.
[clause 164] Use of an enzyme having a kinase activity in the process as defined in any of the previous clauses.
[clause 165] Use of an enzyme having a epimerase activity in the process as defined in any of the previous clauses.
[clause 166] Use of a nucleoside monophosphate to produce a glycosylated organic compound; preferably to produce a glycosylated organic compound according to the process defined in any of the preceding clauses.
[clause 167] The use according to any of clauses 163 or 166, preferably to clause 166, wherein said glycosylated compound is selected from the group consisting of steviol glycosides, polyphenols and oligosaccharides.
[clause 168] The use according to any of clauses 163 to 167, preferably to clause 167, wherein said steviol glycoside selected from the group consisting of steviolmonoside, steviolmonoside A, steviolbioside, steviolbioside D, rubusoside, steviolbioside A, steviolbioside B, rebaudioside B, stevioside, rebaudioside G, stevioside A, stevioside B, stevioside C, rebaudioside A, rebaudioside E, rebaudioside E2, rebaudioside E4, rebaudioside E6, rebaudioside E3, rebaudioside D, rebaudioside I, rebaudioside AM, rebaudioside D7, rebaudioside M, rebaudioside M4, rebaudioside la, rebaudioside lb, rebaudioside lc, rebaudioside Id, rebaudioside le, rebaudioside If, rebaudioside Ig, rebaudioside lh, rebaudioside li, rebaudioside lj, rebaudioside Ik, rebaudioside ll, rebaudioside lm, rebaudioside In, rebaudioside 2a, SvG7 dulcoside A, dulcoside B, dulcoside C, dulcoside D, rebaudioside A2, rebaudioside A3, rebaudioside A4, rebaudioside B2, rebaudioside C, rebaudioside C2, rebaudioside C3, rebaudioside C4, rebaudioside C5, rebaudioside C6, rebaudioside D2, rebaudioside D3, rebaudioside D4, rebaudioside D5, rebaudioside D6, rebaudioside D8, rebaudioside E5, rebaudioside E7, rebaudioside F, rebaudioside F2, rebaudioside F3, rebaudioside H, rebaudioside 112, rebaudioside H3, rebaudioside H4, rebaudioside H5, rebaudioside H6, rebaudioside 12, rebaudioside 13, rebaudioside J, rebaudioside K, rebaudioside K2, rebaudioside KA, rebaudioside L, rebaudioside M2, rebaudioside M3, rebaudioside N, rebaudioside N2, rebaudioside N3, rebaudioside N4, rebaudioside N5, rebaudioside O, rebaudioside O2, rebaudioside O3, rebaudioside O4,rebaudiosideQ, rebaudioside Q2, rebaudioside Q3, rebaudioside R, rebaudioside S, rebaudioside T, rebaudioside Tl, rebaudioside U, rebaudioside U2, rebaudioside V, rebaudioside V2, rebaudioside V3, rebaudioside W, rebaudioside W2, rebaudioside W3, rebaudioside Y, rebaudioside Z1, rebaudioside Z2, steviolbioside C, steviolbioside E, stevioside D, stevioside E, stevioside E2, stevioside F, stevioside G and stevioside H, as set forth in patent literature WO2018213279A1 which is hereby incorporated by reference, synthetic steviol glycosides, and combinations thereof; preferably rebaudioside M.

The following examples further illustrate the invention but are not to be construed as limiting its scope:

### EXAMPLE 1: PREPARATION OF ENZYMES AND ACTIVITY MEASUREMENTS

TABLE 1
reproduces a listing of the sequences SEQ ID NO 1 through 30 used in the process according
to the invention together with the corresponding sequences identifiers and natural sources:

| | | | | UniProtKB/Swiss- | |
|---|---|---|---|---|---|
| | | | | Prot or NCBI Gen- | |
| | | | | Bank or NCBI Ref- | Name in the |
| No. | Enzymatic activity | Type | Organism | Seq | experiments |
| 1 | UMP kinase | wild-type | Escherichia coli (strain K12) | P0A7E9.2 | UMP kinase |
| 2 | sucrose synthase | engineered | variant of | P49040.3 | SuSy_At |
| | | | Arabidopsis thaliana | | PM1-54-2-E05 |
| 3 | sucrose synthase | wild-type | Bambusa oldhamii | AAL50571.1 | SuSy_Bo |
| 4 | UDP- | engineered | variant of | XP_004250485.1 | UGTS1-0234 |
| | glycosyltransferase | | Solanum lycopersicum | | |
| 5 | UDP- | wild-type | Stevia rebaudiana | AGL95113.1 | UGT76G1 |
| | glycosyltransferase 76G1 | | | | |
| 6 | beta-1,4- | wild-type | Neisseria meningitidis | Q51116 | NmLgtB |
| | galactosyltransferase | | serogroup B (strain MC58) | | |
| 7 | UDP-glycosyl- | engineered | variant of | AAR06912.1 | UGTSr- |
| | transferase 76G1 | | Stevia rebaudiana | | 0042 |
| 8 | UDP-D- | wild-type | Arabidopsis thaliana | NP_192834.1 | AtUGE5 |
| | glucose/UDP-D- | | | | |
| | galactose 4- | | | | |
| | epimerase | | | | |
| 9 | sucrose synthase | wild-type | Arabidopsis thaliana | P49040.3 | SuSy_At |
| 10 | UMP kinase | wild-type | Salmonella enterica | NP_459223.1 | S2UMPK |
| 11 | UMP kinase | wild-type | Bacillus subtilis | NP_389533 | BsUMPK |
| 12 | UMP kinase | wild-type | Ureaplasma parvum | WP_006688575 | UpUMPK |
| 13 | UMP kinase | wild-type | Pyrococcus furiosus | WP_011012554 | PfUMPK |
| 14 | UMP kinase | wild-type | Neisseria meningitidis | P65931 | NmUMPK |
| 15 | UMP kinase | wild-type | Streptococcus pyogenes | WP_002985765 | SpUMPK |
| 16 | UMP kinase | wild-type | Haemophilus influenzae | WP_005693391 | HiUMPK |
| 17 | UMP kinase | wild-type | Xanthomonas campestris | WP_011036563 | XcUMPK |
| 18 | UMP kinase | wild-type | Saccharolobus solfataricus | WP_009992427 | SsUMPK |
| 19 | UMP kinase | wild-type | Mycobacterium tuberculosis | WP_003414665 | MtUMPK |
| 20 | UMP kinase | wild-type | Helicobacter pylori | WP_001148056 | HpUMPK |
| 21 | UMP kinase | wild-type | Arabidopsis thaliana | AAB71135 | AtUMPK |
| 22 | UMP kinase | wild-type | Homo sapiens | P30085 | HsUMPK |
| 23 | UMP kinase | wild-type | Saccharomyces cerevisiae | NP_012901 | ScUMPK |
| 34 | UMP kinase | wild-type | Dictyostelium discoideum | XP_637196 | DdUMPK |
| 25 | UMP kinase | wild-type | Gorilla gorilla | XP_030871142.1 | GgUMPK |
| 26 | UMP kinase | wild-type | Rattus norvegicus | NP_001020826 | RnUMPK |
| 27 | UMP kinase | wild-type | Vulpes vulpes | XP_025847664 | VvUMPK |
| 28 | UMP kinase | wild-type | Canis lupus | XP_038543664.1 | ClUMPK |
| 29 | UMP kinase | wild-type | Morchella conica | RPB14989 | McUMPK |
| 30 | UMP kinase | wild-type | Tuber magnatum | PWW76052 | TmUMPK |

### EXAMPLE 1.1.1: PRODUCTION OF UMP KINASE (SEQ ID NO: 1)

UMP kinase with sequence corresponding to SEQ ID NO: 1 (wild-type (UniProtKB/Swiss-Prot: P0A7E9.2); Source: Escherichia coli (strain K12))

Cloning: The gene for wild-type UMP kinase (SEQ ID NO: 1) from Escherichia coli (E. coli) was cloned into the expression vector pLE1A17 (derivative of pRSF-1b, Novagen) containing an N-terminal Strep-tag for purification of UMP Kinase (SEQ ID NO: 1). The resulting plasmid were used for transformation of E. coli BL21(DE3) cells.

Expression: UMP kinase (SEQ ID NO: 1) was expressed by inoculating ZYM505 medium (F. William Studier, Protein Expression and Purification 41 (2005) 207-234) supplemented with kanamycin (50 μg/ml) with a fresh overnight culture. Cultures were grown at 37° C. Expression of the gene was induced at logarithmic phase by IPTG (0.1 mM) and carried out overnight at 30° C.

Preparation and purification of Strep-tagged UMP Kinase (SEQ ID NO: 1): Cells were harvested by centrifugation and suspended in a buffer containing 50 mM Tris-HCl-buffer pH 8, 150 mM NaCl, 2 mM MgCl₂, 0.5 mg/mL lysozyme and 20 U/mL NuCLEANase (c-LEcta GmbH). Cell lysis was achieved by sonication. Cell free extract containing soluble enzyme was separated from the debris by centrifugation. UMP Kinase (SEQ ID NO: 1) was affinity purified from supernatant using Strep-Tactin® Superflow® High Capacity resin (IBA GmbH) and a gravity flow column according to manufacturer's manual The eluted solution was rebuffered into 50 mM potassium phosphate-buffer pH 7 using PD-10 desalting columns according to the manufacturer (GE Healthcare). Aliquots of the purified enzyme solution were shock frozen in liquid nitrogen and stored at −20° C.

Activity measurements: For the determination of the standard activity of UMP Kinase (SEQ ID NO: 1), the synthesis of UDP from UMP and ATP was assayed. An UMP Kinase (SEQ ID NO: 1) unit (U) corresponds to the synthesis of 1 μmol UDP per minute from 1 mM UMP and 1 mM ATP in 50 mM potassium phosphate buffer pH 6.5, 4 mM MgCl2, 1 M sucrose at 45° C. Reaction progress was determined discontinuously by stopping reaction after a given reaction time through addition of 1 volume 54% acetonitrile and quantifying the amount of synthesized UDP via HPLC calibrated with external standard

### EXAMPLE 1.1.2: PRODUCTION OF UMP KINASES (SEQ ID NO: 10 to SEQ ID NO: 30)

UMP kinases with sequence corresponding to SEQ ID NO: 10 to SEQ ID NO: 30

Cloning: The gene for the wild-type UMP kinasess (SEQ ID NO: 10 to SEQ ID NO: 30) from the corresponding organism is cloned into the expression vector pLE1A17(derivative of pRSF-1b, Novagen) containing an N-terminal Strep-tag for purification of UMP Kinases (SEQ ID NO: 10 to SEQ ID NO: 30). The resulting plasmid is used for transformation of BL21(DE3) cells.

Expression: UMP kinases (SEQ ID NO: 10 to SEQ ID NO: 30) is expressed by inoculating ZYM505 medium (F. William Studier, Protein Expression and Purification 41 (2005) 207-234) supplemented with kanamycin (50 μg/ml) with a fresh overnight culture. Cultures are grown at 37° C. Expression of the gene is induced at logarithmic phase by IPTG (0.1 mM) and carried out overnight at 30° C.

Preparation and purification of Strep-tagged UMP Kinases (SEQ ID NO: 10 to SEQ ID NO: 30): Cells are harvested by centrifugation and suspended in a buffer containing 50 mM Tris-HCl-buffer pH 8, 150 mM NaCl, 2 mM MgCl₂, 0.5 mg/mL lysozyme and 20 U/mL NuCLEANase (c-LEcta GmbH). Cell lysis is achieved by sonication. Cell free extract containing soluble enzyme is separated from the debris by centrifugation. UMP Kinases (SEQ ID NO: 10 to SEQ ID NO: 30) is affinity purified from supernatant using Strep-Tactin® Superflow® High Capacity resin (IBA GmbH) and a gravity flow column according to manufacturer's manual The eluted solution is rebuffered into 50 mM potassium phosphate-buffer pH 7 using PD-10 desalting columns according to the manufacturer (GE Healthcare). Aliquots of the purified enzyme solution are shock frozen in liquid nitrogen and stored at −20° C.

Activity measurements: For the determination of the standard activity of UMP Kinases (SEQ ID NO: 10 to SEQ ID NO: 30), the synthesis of UDP from UMP and ATP is assayed. An UMP Kinases (SEQ ID NO: 10 to SEQ ID NO: 30) unit (U) corresponds to the synthesis of 1 μmol UDP per minute from 1 mM UMP and 1 mM ATP in 50 mM potassium phosphate buffer pH 6.5, 4 mM MgCl2, 1 M sucrose at 45° C. Reaction progress is determined discontinuously by stopping reaction after a given reaction time through addition of 1 volume 54% acetonitrile and quantifying the amount of synthesized UDP via HPLC calibrated with external standard

### EXAMPLE 1.2: PRODUCTION OF SuSy_At PM1-54-2-E05 (SEQ ID NO: 2)

SuSy_At PM1-54-2-E05 with sequence corresponding to SEQ ID NO:2 (engineered sucrose synthase; Source of wild-type (UniProtKB/Swiss-Prot: P49040.3): Arabidopsis thaliana)

Cloning: The gene for engineered variant SuSy_At PM1-54-2-E05 (SEQ ID NO: 2) from Arabidopsis thaliana was cloned into the expression vector pLE1A17 (derivative of pRSF-1b, Novagen) The resulting plasmid were used for transformation of E. coli BL21(DE3) cells.

Expression: SuSy_At PM1-54-2-E05 (SEQ ID NO: 2) was expressed by inoculating ZYM505 medium supplemented with kanamycin (50 μg/ml) with a fresh overnight culture. Cultures were grown at 37° C. Expression of the gene was induced at logarithmic phase by IPTG (0.2 mM) and carried out overnight at 30° C.

Preparation of solution of SuSy_At PM1-54-2-E05 (SEQ ID NO: 2): Cells were harvested by centrifugation and suspended in a buffer containing 100 mM potassium phosphate buffer pH 7.0, 2 mM MgCl₂, 0.5 mg/mL lysozyme and 20U/mL NuCLEANase. Cell lysis was achieved by sonication. Cell free extract containing soluble enzyme was separated from the debris by centrifugation. Supernatant was mixed with 1 volume of water and aliquots of the enzyme solution were stored at −20° C.

Activity measurements: For the determination of the standard activity of SuSy_At PM1-54-2-E05 (SEQ ID NO: 2), the synthesis of fructose from sucrose and UDP was assayed. 1 mU of SEQ ID NO: 2 is defined as the amount of enzyme that produces 1 nmol of fructose per minute from 400 mM sucrose and 15 mM UDP in 50 mM potassium phosphate buffer pH 7.0 and 3 mM MgCl₂ at 30° C. Reaction progress was determined discontinuously by quantification of liberated fructose using the Kit K-FRUGL (Megazymes).

Production of SuSy_At wildtype (SEQ ID NO: 9) was done as described for SuSy_At PM1-54-2-E05 (SEQ ID NO: 2).

### EXAMPLE 1.3: PRODUCTION OF PURIFIED SuSy_Bo (SEQ ID NO: 3)

SuSy_Bo with sequence corresponding to SEQ ID NO: 3 (wild-type (NCBI GenBank: AAL50571.1); Source: Bambusa oldhamii)

Cloning: The gene for wild-type SuSy_Bo (SEQ ID NO: 3) from Bambusa oldhamii was cloned into the expression vector pLE1A18 (derivative of pRSF-1b, Novagen) containing an N-terminal 6xHis-tag for purification of SuSy_Bo (SEQ ID NO: 3). The resulting plasmid were used for transformation of E. coli BL21(DE3) cells.

Expression: SuSy_Bo (SEQ ID NO: 3) was expressed by inoculating ZYM505 medium supplemented with kanamycin (50 μg/ml) with a fresh overnight culture. Cultures were grown at 37° C. Expression of the gene was induced at logarithmic phase by IPTG (0.2 mM) and carried out overnight at 18° C.

Preparation and purification of His-tagged SuSy_Bo (SEQ ID NO: 3): Cells were harvested by centrifugation and suspended in a buffer containing 25 mM sodium phosphate buffer pH 7.5, 500 mM NaCl, 2 mM MgCl2, 0.5 mg/mL lysozyme and 20 U/mL NuCLEANase. Cell lysis was achieved by sonication. Cell free extract containing soluble enzyme was separated from the debris by centrifugation and sterile filtration. SuSy_Bo (SEQ ID NO: 3) was affinity purified from supernatant using Ni Sepharose 6 Fast Flow (GE Healthcare) resin (GE Healthcare) and a gravity flow column according to manufacturer's manual The eluted solution containing SuSy_Bo (SEQ ID NO: 3) was 6-fold concentrated by ultrafiltration using centrifugal filter devices. The concentrate was rebuffered into 50 mM potassium phosphate-buffer pH 6.5 using PD-10 desalting columns according to the manufacturer (GE Healthcare). Aliquots of the purified enzyme solution were shock frozen in liquid nitrogen and stored at −20° C.

Activity measurements: For the determination of the standard activity of SuSy_Bo (SEQ ID NO: 3), the synthesis of fructose from Sucrose and UDP was assayed. 1 mU of SuSy_Bo (SEQ ID NO: 3) is defined as the amount of enzyme that produces 1 nmol of fructose per minute from 400 mM sucrose and 22.5 mM UDP in 100 mM potassium phosphate buffer pH 7.0 and 15 mM MgCl₂ at 30° C. Reaction progress was determined photometrically by following continuously the synthesis of fructose through coupled, non-rate-limiting enzyme cascade of the Kit K-FRUGL (Megazymes).

### EXAMPLE 1.4: PRODUCTION OF UGTSl-0234 (SEQ ID NO: 4)

UGTSl-0234 with sequence corresponding to SEQ ID NO:4 (engineered UDP-glycosyltransferase; Source of wild-type (NCBI RefSeq: XP_004250485.1): Solanum lycopersicum).

Cloning: The gene for engineered variant UGTSl-0234 (SEQ ID NO: 4) from Solanum lycopersicum was cloned into the expression vector pLE1A17 (derivative of pRSF-1b, Novagen) The resulting plasmid were used for transformation of E. coli BL21(DE3) cells.

Expression: UGTSl-0234 (SEQ ID NO: 4) was expressed by inoculating ZYM505 medium supplemented with kanamycin (50 μg/ml) with a fresh overnight culture. Cultures were grown at 37° C. Expression of the gene was induced at logarithmic phase by IPTG (0.1 mM) and carried out overnight at 30° C.

Preparation of solution of UGTSl-0234 (SEQ ID NO: 4): Cells were harvested by centrifugation and suspended in a buffer containing 100 mM potassium phosphate buffer pH 7.0, 2 mM MgCl₂, 0.5 mg/mL lysozyme and 20 U/mL NuCLEANase. Cell lysis was achieved by sonication. Cell free extract containing soluble enzyme was separated from the debris by centrifugation. Supernatant was mixed with 1 volume of 1 M sucrose and aliquots of the enzyme solution were stored at −20° C.

Activity measurements: For the determination of the standard activity of UGTSl-0234 (SEQ ID NO: 4), the assay was based on a small-scale biotransformation using 10 mM RebA to be converted to RebD by UGTSl-0234 and a non-limiting amount of sucrose synthase for continuous regeneration of UDP-glucose from sucrose and UDP. 1 mU of UGTSl-0234 (SEQ ID NO: 4) is defined as the amount of enzyme that produces 1 nmol of RebD per minute from 10 mM RebA in 50 mM potassium phosphate buffer pH 7.0, 3 mM MgCl₂, 0.5 M sucrose, 0.25 mM UDP, 3 U/mL SEQ ID NO: 9 at 30° C. Reaction progress was determined discontinuously by stopping reaction after different points in time (e.g. 0, 30, 60, 120 min) and quantifying the amount of synthesized RebD via HPLC analytics calibrated with external standard.

### EXAMPLE 1.5: PRODUCTION OF PURIFIED UGT76G1 (SEQ ID NO: 5)

UGT76G1 with sequence corresponding to SEQ ID NO: 5 (wild-type (NCBI GenBank: AGL95113.1); Source: Stevia rebaudiana)

Cloning: The gene for wild-type UGT76G1 (SEQ ID NO: 5) from Stevia rebaudiana was cloned into the expression vector pLE1A19 (derivative of pRSF-1b, Novagen) containing a C-terminal 6xHis-tag for purification of SEQ ID NO: 5. The resulting plasmid was used for transformation of E. coli BL21(DE3) cells.

Expression: UGT76G1 (SEQ ID NO: 5) was expressed by inoculating ZYM505 medium supplemented with kanamycin (50 μg/ml) with a fresh overnight culture. Cultures were grown at 37° C. Expression of the gene was induced at logarithmic phase by IPTG (0.1 mM) and carried out overnight at 25° C.

Preparation and purification of His-tagged UGT76G1 (SEQ ID NO: 5): Cells were harvested by centrif-ugation and suspended in a buffer containing 25 mM sodium phosphate buffer pH 7.5, 500 mM NaCl, 30 mM imidazole, 2 mM MgCl₂, 0.5 mg/mL lysozyme and 20 U/mL NuCLEANase. Cell lysis was achieved by sonication. Cell free extract containing soluble enzyme was separated from the debris by centrifugation and sterile filtration. UGT76G1 (SEQ ID NO: 5) was affinity purified from supernatant using Ni Sepharose 6 Fast Flow (GE Healthcare) resin (GE Healthcare) and a gravity flow column according to manufacturer's manual The eluted solution containing purified UGT76G1 (SEQ ID NO: 5) was 4-fold concentrated by ultrafiltration using centrifugal filter devices. The concentrate was rebuffered into 50 mM potassium phosphate-buffer pH 6.5 using PD-10 desalting columns according to the manufacturer (GE Healthcare). Aliquots of the purified enzyme solution were shock frozen in liquid nitrogen and stored at −20° C.

Activity measurements: For the determination of the standard activity of UGT76G1 (SEQ ID NO: 5), the synthesis of Resveratrol 3,5-β-di-D-glucoside from Polydatin (Resveratrol 3-β-mono-D-glucoside, piceid) and UDP-glucose was assayed. An UGT76G1 (SEQ ID NO: 5) unit corresponds to the synthesis of 1 μmol resveratrol 3,5-diglucoside per minute from 1 mM Polydatin and 2 mM UDP-glucose in 50 mM potassium phosphate buffer pH 7.0, 50 mM KCl, 0.13 wt % bovine serum albumin, 2 mM MgCl₂ at 30° C. Reaction progress was determined discontinuously by stopping reaction after a given reaction time through addition of 3 volumes 50% acetonitrile including 0.1% trifluoracetic acid and quantifying the amount of synthesized resveratrol 3,5-diglucoside by HPLC calibrated with external standard

### EXAMPLE 1.6: PRODUCTION OF PURIFIED NmLgtB (SEQ ID NO: 6)

NmLgtB with sequence corresponding to SEQ ID NO: 6 (wild-type (UniProtKB/Swiss-Prot: Q51116); Source: Neisseria meningitidis serogroup B (strain MC58))

Cloning: The gene for wild-type NmLgtB (SEQ ID NO: 6) from Neisseria meningitidis was cloned into the expression vector pLE1A18 (derivative of pRSF-1b, Novagen) containing an N-terminal 6xHis-tag and maltose binding protein (MBP) tag for purification and enhanced soluble expression of NmLgtB. The resulting plasmid were used for transformation of E. coli BL21(DE3) cells.

Expression: NmLgtB (SEQ ID NO: 6) was expressed by inoculating ZYM505 medium supplemented with kanamycin (50 μg/ml) with a fresh overnight culture. Cultures were grown at 37° C. Expression of the gene was induced at logarithmic phase by IPTG (0.1 mM) and carried out overnight at 23° C.

Preparation and purification of 6xHis-MBP-tagged NmLgtB (SEQ ID NO: 6): Cells were harvested by centrifugation and suspended in a buffer containing 25 mM sodium phosphate buffer pH 7.4, 500 mM NaCl, 2 mM MgCl₂, 0.5 mg/mL lysozyme and 20 U/mL NuCLEANase. Cell lysis was achieved by sonication. Cell free extract containing soluble enzyme was separated from the debris by centrifugation and sterile filtration. NmLgtB (SEQ ID NO: 6) was affinity purified from supernatant using HisTrap Column with ÄKTAprime (GE Healthcare) according to manufacturer's manual The eluted solution containing NmLgtB (SEQ ID NO: 6) was 5-fold concentrated by ultrafiltration using centrifugal filter devices. The concentrate was rebuffered into 50 mM Tris-HCl buffer pH 7.5 using PD-10 desalting columns according to the manufacturer (GE Healthcare). Aliquots of the purified enzyme solution were shock frozen in liquid nitrogen and stored at −20° C.

Activity measurements: For the determination of the standard activity of NmLgtB (SEQ ID NO: 6), the synthesis of Lacto-N-neotetraose from Lacto-N-triose II and UDP-galactose was assayed. 1 unit of NmLgtB (SEQ ID NO: 6) is defined as the amount of enzyme that produces 1 μmol of LNnT per minute from 5 mM LNTII and 5 mM UDP-galactose in 50 mM Tris-HCl buffer pH 7.5, 2 mM MgCl₂ at 30° C. Reaction progress was determined discontinuously by stopping reaction after a given reaction time through addition of 4 volumes 60% acetonitrile and quantifying the amount of synthesized LNnT via IC (Ion Chromatography) calibrated with external standard

### EXAMPLE 1.7: PRODUCTION OF UGTSr-0042 (SEQ ID NO: 7)

UGTSr-0042 with sequence corresponding to SEQ ID NO: 7 (engineered UDP-glycosyltransferase 76G1; Source of wild-type (NCBI GenBank: AAR06912.1): Stevia rebaudiana).

Cloning: The gene for engineered variant UGTSr-0042 (SEQ ID NO: 7) from Stevia rebaudiana was cloned into the expression vector pLE1A17 (derivative of pRSF-1b, Novagen) The resulting plasmid were used for transformation of E. coli BL21(DE3) cells.

Expression: UGTSr-0042 (SEQ ID NO: 7) was expressed by inoculating ZYM505 medium supplemented with kanamycin (50 μg/ml) with a fresh overnight culture. Cultures were grown at 37° C. Expression of the gene was induced at logarithmic phase by IPTG (0.1 mM) and carried out overnight at 30° C.

Preparation of solution of UGTSr-0042 (SEQ ID NO: 7): Cells were harvested by centrifugation and suspended in a buffer containing 100 mM potassium phosphate buffer pH 7.0, 2 mM MgCl₂, 0.5 mg/mL lysozyme and 20 U/mL NuCLEANase. Cell lysis was achieved by sonication. Cell free extract containing soluble enzyme was separated from the debris by centrifugation. Supernatant was mixed with 1 volume of 1 M sucrose and aliquots of the enzyme solution were stored at −20° C.

Activity measurements: For the determination of the standard activity of UGTSr-0042 (SEQ ID NO: 7), the assay was based on a small-scale biotransformation using 10 mM RebA to be converted to Rebl by UGTSr-0042 (SEQ ID NO: 7) and a non-limiting amount of sucrose synthase for continuous regeneration of UDP-glucose from sucrose and UDP. 1 mU of UGTSr-0042 (SEQ ID NO: 7) is defined as the amount of enzyme that produces 1 nmol of RebI from 10 mM RebA in 50 mM potassium phosphate buffer pH 7.0, 3 mM MglC₂, 0.5 M sucrose, 0.25 mM UDP, 3 U/mL SEQ ID NO: 9 at 30° C. Reaction progress was determined discontinuously by stopping reaction after different points in time (e.g. 0, 30, 60, 120 min) and quantifying the amount of synthesized RebI via HPLC analytics calibrated with external standard.

### EXAMPLE 1.8: PRODUCTION OF PURIFIED EPIMERASE AtUGE5 (SEQ ID NO: 8)

AtUGE5 with sequence corresponding to SEQ ID NO: 8 (wild-type (NCBI RefSeq: NP_192834.1); Source: Arabidopsis thaliana).

Cloning: The gene for wild-type epimerase AtUGE5 (SEQ ID NO: 8) from Arabidopsis thaliana was cloned into the expression vector pLE1A17 (derivative of pRSF-1b, Novagen) containing a C-terminal Strep-tag for purification of AtUGE5 (SEQ ID NO: 8). The resulting plasmid were used for transformation of E. coli BL21(DE3) cells.

Expression: AtUGE5 (SEQ ID NO: 8) was expressed by inoculating ZYM505 medium supplemented with kanamycin (50 μg/ml) with a fresh overnight culture. Cultures were grown at 37° C. Expression of the gene was induced at logarithmic phase by IPTG (0.1 mM) and carried out overnight at 30° C.

Preparation and purification of Strep-tagged AtUGE5 (SEQ ID NO: 8): Cells were harvested by centrifugation and suspended in a buffer containing 50 mM Tris-HCl-buffer pH 8.0, 2 mM MgCl₂, 0.5 mg/mL lysozyme and 20 U/mL NuCLEANase. Cell lysis was achieved by sonication. Cell free extract containing soluble enzyme was separated from the debris by centrifugation and sterile filtration. AtUGE5 (SEQ ID NO: 8) was affinity purified from supernatant using Strep-Tactin® Superflow® High Capacity resin (IBA GmbH) and a gravity flow column according to manufacturer's manual The eluted solution containing purified AtUGE5 (SEQ ID NO: 8) was 5-fold concentrated by ultrafiltration using centrifugal filter devices. The eluted solution was rebuffered into 50 mM Tris-HCl-buffer pH 7.5 using PD-10 desalting columns according to the manufacturer (GE Healthcare). Aliquots of the purified enzyme solution were shock frozen in liquid nitrogen or mixed with 1 volume of glycerol and stored at −20° C.

Activity measurements: For the determination of the standard activity of AtUGE5 (SEQ ID NO: 8), the synthesis of UDP-galactose from UDP-glucose was assayed. 1 unit of AtUGE5 (SEQ ID NO: 8) is defined as the amount of enzyme that produces 1 μmol of UDP-galactose per minute from 1 mM UDP-glucose in 50 mM Tris-HCl buffer pH 7.5 at 30C ° C. Reaction progress was determined discontinuously by stopping reaction after a given reaction time through addition of 1 volume 60% methanol and quantifying the amount of synthesized UDP-galactose via HPLC calibrated with external standard

### EXAMPLE 1.9: PRODUCTION OF SuSy_At PM1-54-2-E05 (SEQ ID NO: 2) IN E. COLI STRAIN K-12 W3110 WITH DELETED GENE ushA

SuSy_At PM1-54-2-E05 is produced as described in EXAMPLE 1.2 with the exception that E. coli strain K-12 W3110 with deleted gene ushA is used for expression instead of strain E. coli BL21(DE3).

### EXAMPLE 1.10: PRODUCTION OF UGTSl-0234 (SEQ ID NO: 4) IN E. COLI STRAIN K-12 W3110 WITH DELETED GENE ushA

UGTSl-0234 is produced as described in EXAMPLE 1.4 with the exception that E. coli strain K-12 W3110 with deleted gene ushA is used for expression instead of strain E. coli BL21(DE3).

### EXAMPLE 1.11: PRODUCTION OF UGTSr-0042 (SEQ ID NO: 7) IN E. COLI STRAIN K-12 W3110 WITH DELETED GENE ushA

UGTSr-0042 is produced as described in EXAMPLE 1.7 with the exception that E. coli strain K-12 W3110 with deleted gene ushA is used for expression instead of strain E. coli BL21(DE3).

### EXAMPLE 2: ONE-POT ENZYMATIC SYNTHESIS OF RebM

### EXAMPLE 2.1: ONE POT ENZYMATIC SYNTHESIS OF RebM USING UDP (UDP-BASED PROCESS)

A reaction solution containing rebaudioside A (RebA) in potassium phosphate buffer, UDP, sucrose and MgCl₂ are reacted with the first glycosyltransferase SuSy_At PM1-54-2-E05 (SEQ ID NO: 2) and the second glycosyltransferase UGTSl-0234 (SEQ-ID NO: 4) for 16 h. Then, the third glycosyltransferase UGTSr-0042 (SEQ ID NO: 7) is added. Samples are taken over time and the amount of RebA, rebaudioside D (RebD), and rebaudioside M (RebM) is determined by HPLC. The amount of rebaudioside species in the sample is quantified using external standards Results are shown in Table 2.

As described in the state of the art, the following typical reaction conditions are used for the process: rebaudioside A (RebA) concentration: from 20 mM up to 100 mM; potassium phosphate buffer from 30 mM up to 100 mM, pH 6-7; UDP: from 0.20 mM up to 1 mM; Sucrose: from 300 mM up to 1 M, MgCl₂ from 2 mM up to 10 mM with reaction temperatures between 40° C. and 55° C. Glycosyltransferases are added with activities between 5 mU/mL and 500 mU/mL.

TABLE 2
Expected formation profile of RebD and RebM for the
one-pot enzymatic synthesis of RebM using UDP (mol
% conversion from RebA start concentration)

| | RebA | RebD | RebM |
|---|---|---|---|
| time/h | (mol %) | (mol %) | (mol %) |
| 0 | 100 | 0 | 0 |
| 16 | 18 | 82 | 0 |
| 20 | 13 | 52 | 34 |
| 24 | 10 | 33 | 55 |
| 30 | 4 | 5 | 87 |
| 42 | 2 | 2 | 93 |

### EXAMPLE 2.2: ONE POT ENZYMATIC SYNTHESIS OF RebM USING UMP, ATP AND UMP KINASE (SEQ ID NO: 1) (UMP-BASED PROCESS)

The reactions described in EXAMPLE 2.1 are repeated, but instead of UDP 0.25-1 mM UMP are employed, and furthermore 0.25-1 mM ATP, and 1-20 mU/mL of UMP kinase (SEQ ID NO: 1) are added to the reaction at reaction start together with SuSy_At PM1-54-2-E05 (SEQ ID NO: 2) and UGTSl-0234 (SEQ-ID NO: 4). after 16 h UGTSr-0042 (SEQ ID NO: 7) is supplemented. Samples are taken over time and the amount of RebA, RebD and RebM is determined by HPLC. It is expected that at starting concentrations of for example 0.4 mM UMP, 0.4 mM ATP and 5 mU/mL UMP Kinase (SEQ ID NO: 1) the reaction leads to similar RebA conversions to RebD and RebM as described in EXAMPLE 2.1 under equal reaction condition.

### EXAMPLE 2.3: ONE POT ENZYMATIC SYNTHESIS OF RebM WITH PURIFIED ENZYMES

RebM synthesis reactions are carried out as described in EXAMPLEs 2.1 and 2.2 with the exception that purified enzymes are used. In order to avoid possible interfering side reactions from production strain E. coli, which may degrade UDP, UMP, ATP and/or UDP-glucose, the enzymes are purified from crude extracts using a biochemical method known in the art, such as precipitation, chromatography or heat purification. It is expected that a lower concentration of UDP (in UDP-based process, e.g. EXAMPLE 2.1) or UMP+ATP (in the UMP-based process e.g. EXAMPLE 2.2) is required to achieve a similar yield of RebM after 30 h in comparison to RebM synthesis reactions using non-purified enzymes.

### EXAMPLE 2.4: ONE POT ENZYMATIC SYNTHESIS OF RebM WITH ENZYMES PRODUCED E. COLI STRAIN K-12 W3110 WITH DELETED GENE ushA

RebM synthesis reactions are carried out as described in EXAMPLEs 2.1 and 2.2 with the exception that enzymes are used which were produced in E. coli strain K-12 W3110 with deleted gene ushA (UDP sugar hydrolase). It is expected that a up to 5-fold lower concentration of UDP (in UDP-based process, e.g. EXAMPLE 2.1) or UMP+ATP (in the UMP-based process e.g. EXAMPLE 2.2) is required to achieve a similar yield of RebM after 30 h in comparison to RebM synthesis reactions using non-purified enzymes.

### EXAMPLE 3: ONE-POT ENZYMATIC SYNTHESIS OF GLC-POLYDATIN (RESVERATROL 3,5-DIGLUCOSIDE AND RESVERATROL 3,5,4′-TRIGLUCOSIDE)

### EXAMPLE 3.1: ONE POT ENZYMATIC SYNTHESIS OF GLC-POLYDATIN USING UDP (UDP-BASED PROCESS)

A reaction solution containing 10 mM polydatin (resveratrol-3-β-mono-D-glucoside, piceid) in 50 mM potassium phosphate buffer pH 6.5, 0.1-0.8 mM UDP, 750 mM sucrose and 2 mM MgCl₂ was preheated to 40° C. and 50 mU/mL SuSy_Bo (SEQ ID NO: 3) and 3.3 mU/ml UGT76G1 (SEQ ID NO: 5) were added. Reactions are incubated at 40° C. shaking at 500 rpm. Samples were taken over time and the amounts of polydatin and Glc-polydatin (resveratrol 3,5-diglucoside and resveratrol 3,5,4′-triglucoside) were quantified by HPLC using external standard Results are shown in Table 3.

TABLE 3
Formation of Glc-Polydatin using 0.125-1 mM UDP (mol
% conversion from polydatin start concentration)

| | 0.1 mM | 0.2 mM | 0.4 mM | 0.8 mM |
|---|---|---|---|---|
| | UDP | UDP | UDP | UDP |
| | Glc- | Glc- | Glc- | Glc- |
| | polydatin | polydatin | polydatin | polydatin |
| time/h | (mol %) | (mol %) | (mol %) | (mol %) |
| 0 | 0 | 0 | 0 | 0 |
| 3 | 6 | 8 | 10 | 10 |
| 21 | 26 | 39 | 50 | 56 |
| 48 | 41 | 60 | 77 | 84 |
| 71 | 48 | 68 | 86 | 91 |

### EXAMPLE 3.2: ONE-POT ENZYMATIC SYNTHESIS OF GLC-POLYDATIN USING UMP, ATP AND SEQ ID NO: 1 (UMP-BASED PROCESS)

A reaction solution containing 10 mM polydatin in 50 mM potassium phosphate buffer pH 6.5, 0.1-0.8 mM UMP, 0.1-0.8 mM ATP, 750 mM sucrose and 2 mM MgCl₂ is preheated to 40° C. and 1-100 mU/mL UMP kinase (SEQ ID NO: 1), 50 mU/mL SuSy_Bo (SEQ ID NO: 3) and 3.3 mU/ml UGT76G1 (SEQ ID NO: 5) are added. Reactions are incubated at 40° C. shaking at 500 rpm. Samples are taken over time and the amounts of polydatin and Glc-polydatin (resveratrol 3,5-diglucoside and resveratrol 3,5,4′-triglucoside) are quantified by HPLC using external standard. It is expected that the formation of Glc-polydatin will proceed in similar conversion levels as in the process using UDP (Example 3.1).

### EXAMPLE 4: ONE-POT ENZYMATIC SYNTHESIS OF LNnT FROM LNTII

### EXAMPLE 4.1: ONE POT ENZYMATIC SYNTHESIS OF LNnT FROM LNTII USING UDP (UDP-BASED PROCESS)

A reaction solution containing 100 mM LNTII (Lacto-N-triose II) in 50 mM Tris-HCl buffer pH 7.5, 1 mM UDP, 500 mM sucrose and 10 mM MgCl₂ was preheated to 30° C. and 550 mU/mL SuSy_Bo (SEQ ID NO: 3), 2300 mU/ml AtUGE5 (SEQ ID NO: 8) and 150 mU/ml NmLgtB (SEQ ID NO: 6) were added. Reactions are incubated at 30° C. shaking at 180 rpm. Samples were taken over time and the amount of LNTII and LNnT (Lacto-N-neotetraose) was quantified by HPLC using external standards. Results are shown in Table 4.

TABLE 4
Formation of LNnT using 1 mM UDP (mol % conversion
from LNTII start concentration)

| | LNTII | LNnT |
|---|---|---|
| time/h | (mol %) | (mol %) |
| 0 | 100 | 1 |
| 1 | 81 | 6 |
| 4 | 84 | 20 |
| 24 | 42 | 51 |
| 48 | 17 | 53 |
| 72 | 11 | 61 |

### EXAMPLE 4.2: ONE POT ENZYMATIC SYNTHESIS OF LNnT FROM LNTII USING UMP, ATP AND UMP KINASE (SEQ ID NO: 1) (UMP-BASED PROCESS)

A reaction solution containing 100 mM LNTII (Lacto-N-triose II) in 50 mM Tris-HCl buffer pH 7.5, 1 2 mM UMP, 1-2 mM ATP, 500 mM sucrose and 10 mM MgCl2 is preheated to 30° C. and 1-200 mU/ml UMP kinase (SEQ ID NO: 1), 550 mU/mL SuSy_Bo (SEQ ID NO: 3), 2300 mU/ml AtUGE5 (SEQ ID NO: 8) and 150 mU/ml NmLgtB (SEQ ID NO: 6) are added. Reactions are incubated at 30° C. shaking at 180 rpm. Samples are taken over time and the amount of LNTII and LNnT (Lacto-N-neotetmose) was quantified by HPLC using external standards It is expected that the formation of LNnT will proceed in similar conversion levels as in the process using UDP (Example 4.1).

### EXAMPLE 5: INFLUENCE OF THE UMP/ATP MOLAR RATIO ON THE RELATIVE CONVERSION DURING SYNTHESIS OF RebM

The reactions described in EXAMPLE 2.2 were performed employing different UMP:ATP molar ratio. UMP was kept at 0.4 mM and the concentration of ATP was varied to have final ratios of UMP:ATP in the reaction mixture ranging from 0.67 to 8. Samples were taken over time and the amount of RebA, RebD and RebM is determined by HPLC. The results are shown in the table below:

TABLE 5
relative conversion at different UMP:ATP ratios. The conversion
corresponding to a UMP:ATP ratio of 1, was taken as reference
reaction for the calculation of the relative conversion:

| | | rel. conversion | ATP-regeneration |
|---|---|---|---|
| Entry | [UMP/ATP] | [%] | system |
| 1 (reference) | 0.67 | 103% | no |
| 2 | 1 | 100% | no |
| 3 | 1.3 | 101% | no |
| 4 | 2 | 89% | no |
| 5 | 4 | 67% | no |
| 6 | 8 | 53% | no |
| 7 (Control) | 0 | 4% | no |

The highest conversion was achieved at a UMP:ATP molar ratio of 1.3 or lower (entries 1, 2 and 3).

### EXAMPLE 6: COMPARATIVE SYNTHESIS OF RebM UNDER CONDITIONS ALLOWING FOR REGENERATION OF THE PHOSPHATE DONOR

The reactions described in EXAMPLE 2.2 were performed using an UMP:ATP ratio of 8 (0.4 mM UMP and 0,05 mM ATP). Additionally 20 U/mL ATP (the phosphate donor), was regenerated by using a pyruvate kinase (from rabbit muscle Type II, ammonium sulfate suspension, 350-600 units/mg Protein, Article P1506 from Merck Germany, formally SigmaAldrich) and 4 mM Phosphoenolpyruvate (PEP, monopotassium salt).

The results are shown in the table below:

TABLE 6
relative conversions achieved in the RebM synthesis using an UMP:ATP
ratio of 8 employing an ATP regeneration system (entry 3a).

| | UMP/ATP | relative conversion | ATP-regeneration |
|---|---|---|---|
| Entry | ratio | [%] | system |
| 3a | 8 | 88% | yes |

The ATP recycling system improves the low conversion caused by the low ATP concentration compared to the reaction without recycling. It is not expected that the recycling reaches the same conversion of a biotransformation as described in EXAMPLE 2.2 with an UMP:ATP ratio of 1.3 or higher.

As it can be concluded by comparing table 5 and 6, the use of a UMP:ATP ratio of comprised between 0.67 and 2 allows for achieving a relative conversion comparable to that which otherwise could only be attained by relying on the use of a ATP-regeneration system (compare Entries 1 to 6 of Table 5 with Entry 3a of Table 6) or higher (Entry 1, Table 5 and Entry 3a of Table 6).

1. A process for the preparation of a glycosylated organic compound by in vitro glycosylation of an organic compound having a nucleophilic group with a saccharide under catalysis of a Leloir glycosyltransferase system comprising at least a first glycosyl transferase and a second glycosyl transferase, the process comprising:
(a) providing a nucleoside monophosphate and a phosphate donor;
(b) reacting the nucleoside monophosphate and the phosphate donor provided in (a) under catalysis of a nucleoside monophosphate kinase thereby obtaining a nucleoside diphosphate;
(c) providing a saccharide donor;
(d) reacting the nucleoside diphosphate obtained in (b) with the saccharide of the saccharide donor provided in (c) under catalysis of the first glycosyl transferase thereby obtaining a nucleoside diphosphate saccharide;
(e) providing the organic compound having a nucleophilic group; and
(f) reacting the nucleoside diphosphate saccharide obtained in (d) with the organic compound having a nucleophilic group provided in (e) under catalysis of at least the second glycosyltransferase thereby obtaining the glycosylated organic compound.

2. The process according to claim 1, wherein the nucleoside monophosphate and the phosphate donor are employed in a molar ratio within the range of from 0.5 to 1.5; preferably in equimolar amount.

3. The process according to claim 1, wherein the phosphate donor differs from the nucleoside monophosphate in a nucleobase and/or in a number of phosphate groups.

4. The process according to claim 1, wherein (a), (b), (c), (d), (e) and (f) are performed in a single reactor.

5. The process according to claim 1, wherein (a), (c) and/or (e) are performed simultaneously.

6. The process according to claim 1, wherein (b), (d) and/or (f) are performed simultaneously.

7. The process according to claim 1, wherein the nucleoside monophosphate is a monophosphorylated conjugate of a ribose or a deoxyribose with uracil; preferably uridine monophosphate.

8. The process according to claim 1, wherein the phosphate donor is a mono- or polyphosphate of a nucleoside; preferably a nucleoside triphosphate; more preferably adenosine triphosphate.

9. The process according to claim 1, wherein the nucleoside monophosphate kinase is selected from the group consisting of UMP-kinases, AMP-kinases, CMP-kinases, GMP-kinases, deoxy-TMP-kinases, deoxy-AMP-kinases, deoxy-CMP-kinases and deoxy-GMP-kinases; preferably an uridylate kinase (UMP-kinase).

10. The process according to claim 1, wherein the nucleoside monophosphate kinase is an uridylate kinase (UMP-kinase) belonging to EC class 2.7.4.22.

11. The process according to claim 1, wherein the nucleoside monophosphate kinase is an uridylate kinase (UMP-kinase) belonging to EC class EC 2.7.4.14.

12. The process according to claim 1, wherein the nucleoside monophosphate kinase is is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30.

13. (canceled)

14. The process according to claim 1, wherein the first glycosyl transferase is a sucrose synthase; preferably a uridine diphosphate specific sucrose synthase.

15. The process according to claim 1, wherein the second glycosyltransferase is selected from the group consisting of uridine diphosphate glycosyltransferases, adenosine diphosphate glycosyltransferases, cytidine diphosphate glycosyltransferase, guanosine diphosphate glycosyltransferase, thymidine diphosphate glycosyltransferase; preferably an uridine diphosphate dependent glycosyltransferase.

16. The process according to claim 1, wherein the process involves in step (f) the use of a third glycosyltransferase and wherein said third glycosyltransferase is a nucleotide sugar-dependent glycosyltransferase.

17. The process according to claim 1, wherein the nucleoside diphosphate saccharide obtained in (d) comprises a sugar moiety and wherein the process comprises the further (h) converting the sugar moiety into an epimer thereof under catalysis of an epimerase.

18. The process according to claim 1, wherein at least one enzyme provided has been produced in a genetically modified organism.

19. The process according to claim 18, wherein the genetically modified organism comprises a genetic modification involving deletion of one or more genes encoding for a polypeptide having nucleotide diphosphate-sugar hydrolase activity.

20. The process according to claim 19, wherein said hydrolase activity is a 5′-nucleotidase or UDP-sugar hydrolase activity.

21. (canceled)

22. The process according to claim 1, wherein
the glycosylated organic compound is rebaudioside M (reb M); and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is employed in a concentration of at least 1.5 mU/ml; preferably of at least 4.5 mU/ml; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 2; and/or
the nucleoside diphosphate saccharide is uridine diphosphate glucose (UDP-glucose); and/or
the organic compound having a nucleophilic group is rebaudioside A (reb A); and/or
the organic compound having a nucleophilic group is employed a concentration of about 40 mM; and/or
the second glycosyltransferase is an uridine diphosphate dependent glycosyltransferases (UDP-glycosyltransferases) comprising a primary sequence having an having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 4; and/or
the third glycosyltransferase is an uridine diphosphate dependent glycosyltransferases (UDP-glycosyltransferases) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 7; and/or
the phosphate donor and the nucleoside monophosphate are employed in a total concentration within the range 0.5 mM to 2.0 mM; and/or
the nucleoside monophosphate and the phosphate donor are employed in a molar ratio within the range 0.6 o 1.5, such as 1.0±0.5, and most preferably the nucleoside monophosphate and the phosphate donor are employed in equimolar amount, i.e. in a molar ratio of 1.0; and/or
the process is carried out at a temperature of around 45° C.; and/or
the process is carried out at a pH of about 6.5; and/or
the process is carried out within a total reaction time of about 41.5 h; and/or
the saccharide donor is added to the reaction in a concentration of about 1000 mM; and/or
in (b) the phosphate donor is not regenerated.

23. The process according to claim 1, wherein
the glycosylated organic compound is Glc-polydatin; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the organic compound having a nucleophilic group is polydatin; and/or
the organic compound having a nucleophilic group is employed a concentration of about 10 mM; and/or
the second glycosyltransferase is an uridine diphosphate dependent glycosyltransferase (UDP-glycosyltransferase) comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 5; and/or
the phosphate donor and the nucleoside monophosphate are employed in a total concentration within the range 0.2 mM to 2 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0;and/or
the process is carried out at a temperature of around 40° C.; and/or
the process is carried out at a pH of about 6.5; and/or
the process is carried out within a total reaction time of about 71 h; and/or
the saccharide donor is added to the reaction in a concentration of about 750 mM and/or
in (b) the phosphate donor is not regenerated.

24. The process according to claim 1, wherein
the glycosylated organic compound is Lacto-N-neotetraose; and/or
the nucleoside monophosphate is uridine monophosphate (UMP) and the nucleoside diphosphate is uridine diphosphate (UDP); and/or
the phosphate donor is adenosine triphosphate (ATP); and/or
the nucleoside monophosphate kinase is an uridylate kinase (UMP-kinase) consisting of an amino acid sequence selected from the group of sequences consisting of SEQ ID: NO:1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30; and/or
the nucleoside monophosphate kinase is an uridylate kinase (UMP-kinase) belonging to EC class EC 2.7.4.1; and/or
the nucleoside monophosphate kinase is an uridylate kinase (UMP-kinase) belonging to EC class 2.7.4.22; and/or
the nucleoside monophosphate kinase is an uridylate kinase (UMP-kinase) comprising a primary sequence having a sequence identity of at least 70% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the nucleoside monophosphate kinase is an uridylate kinase (UMP-kinase) comprising a primary sequence having a sequence identity of at least 90% to SEQ ID NO: 1, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and/or SEQ ID NO: 30; and/or
the saccharide donor is sucrose; and/or
the first glycosyl transferase is a uridine diphosphate specific sucrose synthase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 3; and/or
the nucleoside diphosphate saccharide is uridine diphosphate glucose (UDP-glucose); and/or
the glucose moiety of nucleoside diphosphate saccharide is converted into a galactose moiety under catalysis of a glucose galactose epimerase wherein the glucose galactose epimerase is UDP-glucose 4-epimerase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and in particular of at least 98%, or at least 99%, or 100%; preferably of at least 85%, or at least 86%, or at least 87%, or at least 88%, or at least 89%, or at least 90%, or at least 91%, or at least 92%, or at least 93%, or at least 94%, or at least 95%, or at least 96%, or at least 97%; and in particular at least 98%, or at least 99%, or 100%, in each case to SEQ ID NO: 8; and/or
the organic compound having a nucleophilic group is Lacto-N-triose II; and/or
the organic compound having a nucleophilic group is employed a concentration of about 100 mM; and/or
the second glycosyltransferase is a galactosyltransferase; preferably a beta-1,4-galactosyltransferase comprising a primary sequence having a sequence identity of at least 60%, or at least 61%, or at least 62%, or at least 63%, or at least 64%, or at least 65%, or at least 66%, or at least 67%, or at least 68%, or at least 69%, or at least 70%, or at least 71%, or at least 72%, or at least 73%, or at least 74%, or at least 75%, or at least 76%, or at least 77%, or at least 78%, or at least 79%, or at least 80%, or at least 81%, or at least 82%, or at least 83%, or at least 84%, or at least 85%, or at least 86%, or at least 87%, or at least 88%, or at least 89%, or at least 93%, or at least 94%, at least 95%, or at least 96%, or at least 97%; and
in particular at least 98%, or at least 99%, or 100%; preferably of at least 85%, or at least 86%, or at least 87%, or at least 88%, or at least 89%, or preferably at least 93%, or at least 94%, or at least 95%, or at least 96%, or at least 97%; and in particular at least 98%, or at least 99%, or 100%, in each case to SEQ ID NO: 6; and/or
the phosphate donor and the nucleoside monophosphate are employed in a total concentration within the range 2 mM to 4 mM; and/or
the nucleoside monophosphate and the phosphate donor are employed in a molar ratio within the range 0.6 to 1.5, such as 1.0±0.5, and most preferably the nucleoside monophosphate and the phosphate donor are employed in equimolar amount, i.e. in a molar ratio of 1.0; and/or
the process is carried out at a temperature of around 30° C.; and/or
the process is carried out at a pH of about 7.5; and/or
the process is carried out within a total reaction time of about 72 h; and/or
the saccharide donor is added to the reaction in a concentration of about 500 mM; and/or
in (b) the phosphate donor is not regenerated.

25. A composition comprising a (i) glycosylated organic compound obtainable by the process according to claim 1 in combination with (ii) a nucleoside monophosphate kinase, or a first glycosyltransferase, a second glycosyltransferase, or a third glycosyltransferase an epimerase, or any combination of the foregoing.

26. (canceled)

27. (canceled)

28. (canceled)

29. A method comprising:
providing an enzyme having a glycosyltransferase activity, wherein the enzyme is part of a Leloir glycosyltransferase system comprising at least a first glycosyl transferase and a second glycosyl transferase and catalyzes a reaction of (i) a nucleoside diphosphate with a saccharide of a saccharide donor to obtain a nucleoside diphosphate saccharide or (ii) the nucleoside diphosphate saccharide obtained in (i) with a nucleophilic group of an organic compound to obtain a glycosylated organic compound, wherein the nucleoside diphosphate in (i) is obtained by a reaction of a nucleotide monophosphate with a phosphate donor, wherein the reaction is catalyzed via an nucleoside monophosphate kinase according to claim 1.

30. A method comprising:
providing an enzyme having a kinase activity, wherein the enzyme catalyzes a reaction of a nucleoside monophosphate and a phosphate donor to obtain a nucleoside diphosphate, wherein the nucleoside diphosphate is further reacted with a saccharide of a saccharide donor via a first glycosyl transferase of a Leloir glycosyltransferase system to obtain a nucleoside diphosphate saccharide, which in turn is further reacted with a organic compound having a nucleophilic group via a second glycosyl transferase of the Leloir glycosyltransferase system to obtain a glycosylated organic compound according to claim 1.
